(19) United States
(12) Patent Application Publication
(10) Pub. No.: US 2008/0216840 A1
(43) Pub. Date: Sep. 11, 2008

(54) Imaging via the airway

(75) Inventors: Edward S. Boyden, Cambridge, MA (US); Roderick A. Hyde, Redmond, WA (US); Muriel Y. Ishikawa, Livermore, CA (US); Eric C. Leuthardt, St. Louis, MO (US); Nathan P. Myhrvold, Medina, WA (US); Dennis J. Rivet, St. Louis, MO (US); Michael A. Smith, Phoenix, AZ (US); Clarence T. Tegreene, Bellevue, WA (US); Thomas A. Weaver, San Mateo, CA (US); Charles Whitmer, North Bend, WA (US); Lowell L. Wood, Bellevue, WA (US); Victoria Y.H. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/715,500

(22) Filed: Mar. 6, 2007

(51) Int. Cl. A61M 16/00

(52) U.S. Cl. 128/207.14

(57) ABSTRACT

Embodiments include a medical device, an apparatus, and a method. The medical device includes a tube portion shaped and sized for airway passage insertion and having at least one pathway therethrough; and a stylet of at least one shape-transforming material, configured for insertion in one of said at least one pathway; and at least one sensor carried by the tube portion and the stylet. The apparatus includes a stylet having a first end and a second end, at least a portion of the stylet including a shape-transforming material, the stylet being configured for insertion through an airway intubation structure having at least one pathway therethrough; and at least one sensor carried by the stylet in a proximity to the first end. The method includes capturing an image adjacent to an airway tube proximate to an airway passage; and actuating, in response to the image, at least one shape-transformation of the airway tube.

### TECHNICAL FIELD

The present application relates, in general, to devices, methods and/or systems for treatment and/or management of disease, disorders, or conditions.

### SUMMARY

An embodiment of a medical device comprises a tube portion shaped and sized for airway passage insertion. At least one tube has at least one pathway therethrough. Furthermore, the medical device comprises a stylet of at least one shape-transforming material, configured for insertion in the tube pathway, and at least one sensor that is carried by a tube portion and/or the stylet. In an embodiment, a tube portion includes at least one pathway therethrough. Furthermore, a tube portion includes at least one pathway for insertion of at least one stylet. In another embodiment, a tube portion includes at least one pathway for insertion of at least one stylet that has at least one sensor. In yet another embodiment, a tube portion includes at least one pathway for insertion of at least one stylet that has at least one sensor disposed anywhere other than at the ends of the stylet, but supported by an intermediate portion of the stylet. Additionally, a tube portion includes at least one pathway for insertion of at least one stylet that has at least one sensor that is located in a proximity to one or the other end of the stylet. In another embodiment, a tube portion includes at least one pathway for insertion of at least one stylet having multiple sensors.

In an embodiment, the medical device provides for at least one sensor, which includes an image-acquisition device. In a further embodiment, the medical device includes at least one data-transmission device disposed either in or outside a tube portion. The data-transmission device is configured to operatively communicate with at least one sensor. In yet another embodiment, at least one data-transmission device includes an image-transmission device, which may be disposed either in or outside a stylet. The data-transmission device is configured to operatively communicate with at least one sensor. The stylet includes a data-transmission device that may include an image-transmission device. In an embodiment, an image-acquisition device in the medical device includes at least one of a camera, a charge coupled device, an X-ray receiver, an acoustic energy receiver, an electromagnetic energy receiver and/or an imaging device. In a different embodiment, the image-acquisition device is wirelessly coupled to at least one visual display.

In another embodiment, the medical device further comprises a source of illumination, which may be operably coupled to at least one image acquisition device. In a further embodiment, the source of illumination may be located internally within a living body and/or may be located external to a living body. Furthermore, the source of illumination includes at least one of an ultrasonic source, an acoustic source, a visible source, an ultraviolet source, a gamma ray source, an X-ray source and/or an infrared source.

In yet another embodiment, a tube portion in the medical device includes at least one data-transmission device that includes at least one of an optical fiber, a nanotube, a metal wire and/or a nonmetallic wire. In another embodiment, the medical device includes at least one stylet that provides for at least one data-transmission device, which includes at least one of an optical fiber, a metal wire, a nanotube and/or a nonmetallic wire.

In some embodiments, the medical device includes a tube portion having at least one data-transmission device that is adapted to pass electromagnetic, optical, microwave and/or acoustic energy. In another embodiment, the medical device includes a stylet having at least one data-transmission device that is adapted to pass electromagnetic, optical, microwave and/or acoustic energy.

In yet another embodiment, the medical device includes a tube portion having at least one data-transmission device that is adapted to pass at least a signal, a datum, an image and/or a model. In another embodiment, the medical device includes a stylet having at least one data-transmission device that is adapted to pass at least a signal, a datum, an image and/or a model.

In a further embodiment, the medical device has at least one image-acquisition device that is configured to operably communicate with at least one visual display. The visual display may include at least one of an electronically-activatable display screen and/or a chemically-activatable display surface. In a further embodiment, the visual display is coupled to the image-transmission device, which includes a wireless device placed in a tube portion and/or in the stylet. In an embodiment the image-transmission device is placed either in a tube portion and/or the stylet of the medical device that includes a conduit and/or channel.

In one embodiment the medical device includes at least one visual display, which may be coupled to an image-transmission device. The image-transmission device includes a wireless device.

In another embodiment, a tube portion of the medical device is pliable and/or malleable and/or rigid and/or deformable and/or disposable and/or reusable. In yet another embodiment, a stylet of the medical device is pliable and/or malleable and/or rigid and/or deformable and/or disposable and/or reusable. In a further embodiment, a tube portion of the medical device is removable from other parts of the medical device. In another embodiment, at least some portions of a tube portion of the medical device are either cylindrically shaped or non-cylindrically shaped. In yet another embodiment, at least a portion of the stylet of the medical device is either cylindrically shaped or non-cylindrically shaped and is configured and/or shaped to fit into a tube portion. In a further embodiment, the medical device includes at least one image-acquisition device that is disposed inside or outside the stylet. In a further embodiment, the medical device includes at least one image-acquisition device that is disposed inside or outside a tube portion. In another embodiment of the medical device, a tube portion is operatively configured for functioning independently of other components of the medical device. Likewise in other embodiments, the stylet is removable from other parts of the medical device.

In a further embodiment, the medical device includes at least one sensor that is disposed inside or outside either a tube portion or a stylet of the medical device. In another embodiment, at least one sensor is located in proximity to either one or the other end of the stylet. In a further embodiment, the medical device includes at least one sensor that is disposed inside or outside a tube portion, and at least one sensor is located in proximity to either one or the other end of a tube portion. In another embodiment at least one sensor in a tube portion of the medical device is operably coupled to a data-transmission device. In some embodiments, the data-transmission device is operably coupled to at least one visual display. In yet another embodiment, the data-transmission device is operably coupled to at least one audio signal generator. In an embodiment, at least one sensor in a stylet of the medical device is operably coupled to a data-transmission device. In other embodiments, the data-transmission device is operably coupled to at least one visual display. In yet another embodiment, the data-transmission device is operably coupled to at least one audio signal generator. In a further aspect of the medical device at least one visual display is located in physical proximity to a tube portion and/or in proximity to the stylet. In a further aspect of the medical device, at least one visual display is remotely located relative to a tube portion. In another embodiment, at least one visual display is remotely located relative to a stylet.

In one embodiment at least one visual display is mounted on a tube portion of the medical device. In another embodiment at least one visual display is mounted on a stylet of the medical device. In yet another embodiment, the stylet includes one or more lumens and/or channels that are configured for insertion of one or more airway imaging modalities or imaging devices.

In an embodiment of the medical device, a stylet is integrally formed with or is made from at least one shape-transforming material. In another embodiment of the medical device, at least one shape-transforming material is made from a shape memory alloy. The shape memory alloy includes at least one of titanium, nickel, zinc, copper, aluminum, cadmium, platinum, iron, manganese, cobalt, gallium and/or tungsten. In one approach, the shape memory alloy includes Nitinol™ and/or an electro-active polymer and/or at least one mechanically reconfigurable material and/or an electrically conductive material.

In one embodiment, a stylet is guided and /or actuated through at least one pathway in a tube by an application of a voltage profile and/or a temperature profile and/or a magnetic force field and/or steering force and/or pressure profile and/or an electrical current. In another embodiment, a stylet is guided and/or actuated through a pathway by a solid-state phase change. In another embodiment, a stylet is guided and /or actuated through a pathway in a tube by interaction with at least a portion of a wall of the pathway in the tube or by interaction with at least a portion of a wall of the of the tube in response to separation between the portion of the stylet and at least a portion of the wall of the tube.

One embodiment of the medical device further comprises a power source, which is either is mounted on the medical device and/or is configured to deliver power from a remote location relative to the medical device.

In one embodiment, a tube portion of the medical device is shaped and sized for airway passage insertion. In a further embodiment, a tube portion is configured for co-navigation through an airway passage in a human or an animal. The airway includes, but is not limited to, at least one of a nasal cavity, an entrance to a visceral tract, a pharynx, a trachea, a larynx, a nares, a mouth, a sinus, a oropharynx, a bronchus, a bronchiole, an alveolus, an entrance to a respiratory tract, a stoma, a ventilator, a tracheostomy and/or a cricothyroidotomy.

Another aspect provides for an apparatus that includes a stylet having a first end and a second end and at least a portion of the stylet includes a shape-transforming material. The stylet is configured for insertion through an airway intubation structure, which has at least one pathway therethrough, and at least one sensor is carried by the stylet. In one embodiment, the airway intubation structure includes a plurality of pathways therethrough. A further embodiment includes an airway intubation structure that has at least one pathway for insertion of at least one stylet. Additionally, the airway intubation structure includes at least one pathway for insertion of at least one stylet that has at least one sensor. In yet another embodiment, the airway intubation structure includes at least one pathway for insertion of at least one stylet having at least one sensor disposed anywhere other than at the ends of the stylet, but supported by an intermediate portion of the stylet. In a different embodiment, the airway intubation structure includes at least one pathway for insertion of at least one stylet having at least one sensor that is located in a proximity to one or the other end of the stylet. The airway intubation structure may include at least one pathway for insertion of at least one stylet that has multiple sensors.

In another embodiment, the apparatus further comprises a stylet having a source of illumination, which may be operably coupled to at least one image acquisition device. In a further embodiment, the source of illumination may be located internally within a living body and/or may be located external to a living body. Furthermore, the source of illumination includes at least one of an ultrasonic source, an acoustic source, a visible source, an ultraviolet source, a gamma ray source, an X-ray source and/or an infrared source.

In an embodiment, the apparatus further comprises at least one stylet having a suction device for aspirating visualized secretions/mucus plugs, and for irrigating and/or for cleaning a visualization apparatus or a lens.

In some embodiments, the apparatus comprises at least one sensor that includes an image-acquisition device. In yet another embodiment, the medical device has at least one data-transmission device disposed within or outside a stylet. Furthermore, at least one data-transmission device is configured to operatively communicate with at least one sensor. In another embodiment the medical device has at least one data-transmission device that includes an image-transmission device, which is operably coupled to at least one audio signal generator. In a further embodiment, the apparatus includes at least one data-transmission device that is operably coupled to at least one visual display. In an alternative embodiment, at least one data-transmission device is a wireless device that is either disposed in a conduit and/or channel or is disposed outside the channel or conduit.

In a further embodiment, the apparatus comprises an airway intubation structure that is an endotracheal tube. In some embodiments, the apparatus comprises a stylet that is configured to fit into the airway tube. In another embodiment, the stylet in the apparatus is either cylindrically shaped or non-cylindrically shaped and is adapted to facilitate insertion of the airway intubation structure into a human patient or an animal. In another embodiment, the stylet of the apparatus carries at least one sensor that is disposed either inside or outside of the stylet. In yet another embodiment, the sensor is disposed either inside or outside of an airway intubation structure. In further embodiment, the stylet of the apparatus carries at least one sensor that is located in a proximity to the first end of the stylet. In yet another embodiment, the stylet of the apparatus carries at least one data transmission device that is located in proximity to the first end of the stylet. In a further embodiment, at least one visual display is located in a physical proximity to the airway intubation structure and/or is located in a physical proximity to the stylet. In an alternative embodiment, at least one visual display is remotely located relative to the airway intubation structure and/or the stylet. In yet another alternative embodiment, at least one visual display is mounted on the stylet and/or the airway intubation structure.

In a further embodiment, a stylet of the apparatus includes one or more lumen and/or channels, which are configured for insertion of one or more airway imaging modalities or imaging devices. In another embodiment, the stylet of the apparatus is integrally made from shape-transforming material and/or carries a shape-transforming material, which is disposed within a lumen and/or channel of the stylet. Alternatively, the stylet is made from a shape memory alloy. In a further embodiment, the shape-transforming material is made from an electro-active polymer and/or at least one mechanically reconfigurable material. In an alternative embodiment, the shape-transforming material includes a shape memory alloy. The shape memory alloy may include at least one of titanium, nickel, zinc, copper, aluminum, cadmium, platinum, iron, manganese, cobalt, gallium and/or tungsten and/or Nitinol™. In an alternative embodiment, the stylet in the apparatus further comprises a plurality of shape-transforming materials of differing shape-transition characteristics. In certain embodiments of the stylet, the plurality of shape-transforming materials that are made from of differing shape memory alloys that respond to differing transition temperatures and/or electrical properties. Another embodiment of the stylet in the apparatus further comprises a temperature-imparting medium, which may be configured to responds to differing transition temperatures. In a further embodiment, the stylet is guided and/or actuated during the insertion by a solid-state phase change. In some embodiments, the stylet in the apparatus has a plurality of shape-transforming materials, which are independently actuatable by differing transition temperatures and/or electrical inputs. In an alternative embodiment, the plurality of shape-transforming materials controls a shape of the stylet upon actuation of the plurality of shape-transforming materials via temperature and/or electrical inputs. In another embodiment the apparatus includes at least one temperature-imparting medium, which responds to differing transition temperatures.

In an embodiment, the apparatus further comprises one or more processors. In some embodiments, at least one of the processors is an image-processor. In other embodiments, a stylet in the apparatus is configured to carry at least one of the one or more processors. In a further embodiment, the apparatus comprises an airway intubation structure that is configured to carry at least one of the one or more processors. In some embodiments, at least one of the one or more processors is located in a proximity to a first end and/or a second end of a stylet in the apparatus.

In yet another embodiment, the apparatus includes at least one image processor that is operatively coupled to at least one visual display. In a further embodiment, one or more image processors are operatively coupled to at least one audio generator. In other embodiments, the apparatus includes an image processor that is operatively coupled to a visual display configured to be mounted in proximity to a second end of the stylet. In other embodiments, the image processor is operatively coupled to at least one remotely located visual display. In an alternative embodiment, an airway structure of the apparatus is configured to carry at least one of the one or more processors.

In an embodiment, the apparatus further includes a wireless transmitter and/or a wireless receiver and/or transceiver. In yet another embodiment, the apparatus includes at least one sensor that is an acoustic sensor and/or an air-flow monitor. In a further embodiment, the air-flow monitor employs externally-supplied gas-flow, in which a portion of the externally supplied gas-flow is used to provide power to the apparatus.

In an embodiment, the apparatus includes at least one sensor, which includes a gas monitor and/or an oxygen sensor, and/or a CO₂ sensor and/or a temperature sensor and/or pressure sensor and/or a water sensor and/or a chemical sensor.

In yet another embodiment, the apparatus includes at least one electric field receiver and/or an electromagnetic radiation receiver and/or a magnetic field receiver and/or an acoustic receiver. In a further embodiment, the apparatus is configured to deliver energy including audio, ultrasonic, acoustic, visible, ultraviolet, gamma rays, X-rays and/or infrared. In a further embodiment, the apparatus is configured to detect energy including audio, ultrasonic, acoustic, visible, ultraviolet, gamma rays, X-rays and/or infrared.

In one embodiment, the apparatus includes at least one or more processors that are configured to process one or more input signals that include one or more digital or analogue signals. In a further embodiment, at least one of the one or more processors is configured to transmit at least one signal. In an alternative embodiment, a stylet of the apparatus is configured to carry at least one of a receiver, a transceiver and/or a transmitter. In yet another alternative embodiment, at least one receiver is configured to receive digital or analogue input signals from a remote source. In another embodiment, the apparatus includes at least one transmitter and/or at least one transceiver that are configured to transmit digital or analog output signals to a remote receiver. Alternatively, at least one receiver or transmitter or transceiver is configured to transmit digital or analogue signals to at least one visual display. One embodiment calls for the apparatus to include at least one receiver or transmitter or transceiver which is configured to transmit digital or analogue signals to at least one audio generator.

In a further embodiment, a stylet of the apparatus is configured to supply a drug, a pharmaceutical, a nutraceutical, a chemical agent and/or an anesthetic substance into an airway of a living body.

In another embodiment, the apparatus includes a power source, which may be either mounted on the apparatus or is configured to deliver power from a remote location relative to the apparatus.

In yet another embodiment, a stylet has a first end and a second end, and at least a portion of the stylet includes a shape-transforming material. The stylet is configured for insertion through an airway intubation structure having at least one pathway therethrough. Furthermore, the stylet is configured for co-navigation through the airway intubation structure.

In yet another embodiment, the apparatus may be used to image via an airway that includes, but is not limited to, at least one of a nasal cavity, an entrance to a visceral tract, a pharynx, a trachea, a larynx, a nares, a mouth, a sinus, a oropharynx, a bronchus, a bronchiole, an alveolus, an entrance to a respiratory tract, a stoma, a ventilator, a tracheostomy and/or a cricothyroidotomy.

In another aspect, a tube has at least one pathway therethrough and the tube is made from at least one shape-transforming material. Additionally, the tube is configured for airway passage insertion. Furthermore, the tube carries at least one sensor. At least one sensor is remotely located outside a living body but is operably coupled to at least one of an image transmission device, an image acquisition device and/or a data-transmission device. In an embodiment, the tube includes a plurality of pathways therethrough. In another embodiment, the tube includes at least one pathway for insertion of at least one stylet. Furthermore, the tube includes at least one pathway for insertion of at least one stylet having at least one sensor. In a different embodiment, the tube includes at least one pathway for insertion of at least one stylet having at least one sensor disposed anywhere other than at the ends of the stylet, but supported by an intermediate portion of the stylet. In yet another embodiment, the tube includes at least one pathway for insertion of at least one stylet having at least one sensor that is located in a proximity to one or the other end of the stylet. The tube may include at least one pathway for insertion of at least one stylet having multiple sensors.

In another embodiment, the apparatus further comprises a tube having a source of illumination, which may be operably coupled to at least one image acquisition device. In a further embodiment, the source of illumination may be located internally within a living body and/or may be located external to a living body. Furthermore, the source of illumination includes at least one of an ultrasonic source, an acoustic source, a visible source, an ultraviolet source, a gamma ray source, an X-ray source and/or an infrared source.

In one embodiment of the apparatus, at least one sensor includes an image-acquisition device. Some embodiments provide that the apparatus include at least one data-transmission device disposed in or outside the tube, the data-transmission device being configured to operatively communicate with at least one sensor. In an alternative embodiment, at least one data-transmission device includes an image-transmission device.

In an embodiment of the apparatus, the tube is an airway tube for insertion in a body lumen. In a further embodiment, a tube in the apparatus is pliable, and malleable, and is made from an electromagnetically responsive material. In an alternative embodiment, the tube is made at least one shape-transforming material that includes a shape memory alloy. Furthermore, the shape memory alloy may include at least one of titanium, nickel, zinc, copper, aluminum, cadmium, platinum, iron, manganese, cobalt, gallium and/or tungsten. In an embodiment, a tube in the apparatus includes a shape memory alloy that includes Nitinol™ and/or an electro-active polymer and/or at least one mechanically reconfigurable material.

In some embodiments, the airway insertion is guided and/or actuated in a body lumen by the application of a voltage profile and/or a temperature profile and/or by a magnetic force field or by the application of voltage or current and/or by the application of a pressure profile. In other embodiments, airway insertion is guided and/or actuated during the insertion by a solid-state phase change.

In a further embodiment, a tube in the apparatus includes one or more actuatable cuffs and/or sleeves that may be circumferentially disposed on the tube. In a further embodiment, at least one actuatable cuff and/or sleeve engages a luminal wall of a trachea upon inflation of the cuff or sleeve. In an embodiment of the apparatus at least one of the one or more actuatable cuff and/or sleeves is actuatable upon application of voltage or current, and/or pressure. In another embodiment, the apparatus includes at least one sensor that is mounted on at least one actuatable cuff and/or sleeve.

In a further embodiment a tube in the apparatus includes at least one receiver, at least one of which is configured to communicate with at least one sensor. In a further embodiment, a tube in the apparatus includes an acoustic sensor an oxygen sensor, a CO₂ sensor, a temperature sensor, a pressure sensor, a water sensor and/or a chemical sensor. An embodiment of the apparatus provides a magnetic field receiver, an acoustic receiver, an electric field receiver and/or an electromagnetic radiation receiver.

An embodiment of the apparatus contains an air-flow monitor. In a further embodiment, the apparatus includes an air-flow monitor that employs externally supplied gas-flow. In yet another embodiment of the apparatus, a portion of the externally supplied air-flow is used to provide power to the apparatus.

In another embodiment, the tube in the apparatus includes at least one of a receiver or a transceiver or a transmitter. In a further embodiment the transmitter is a wireless transmitter, a receiver and/or the transceiver is a wireless receiver and/or wireless transceiver. In one embodiment, the receiver and/or transceiver are configured to deliver energy including audio, ultrasonic, acoustic, visible, microwave, gamma rays, X-rays, ultraviolet and/or infrared. In another embodiment, the receiver and/or transceiver are configured to detect energy including audio, ultrasonic, acoustic, visible, microwave, gamma rays, X-rays, ultraviolet and/or infrared. In an embodiment, the receiver and/or transceiver are configured to receive digital or analogue input signals and process the input signals. In a further embodiment, the transmitter is configured to transmit visual and/or audio signals. Another embodiment provides that a receiver is configured to transmit input signals to a visual display or an audio generator.

In an embodiment, the apparatus contains a tube comprising a plurality of shape-transforming materials having differing shape-transition characteristics. In a further embodiment, the plurality of shape-transforming materials is made from differing shape memory alloys. In yet another embodiment, the plurality of shape-transforming materials responds to differing transition temperatures and/or electrical inputs. In a further embodiment, the plurality of shape-transforming materials is independently actuatable by differing transition temperatures and/or electrical inputs. Another embodiment provides that the plurality of shape-transforming materials control a shape of a tube in the apparatus upon actuation of the plurality of shape-transforming materials via temperature and/or electrical inputs. An embodiment of the apparatus further comprises a temperature-imparting medium wherein the temperature-imparting medium responds to differing temperature characteristics.

The apparatus in some embodiments further contains a power source. In other embodiments, the power source is mounted on the apparatus or is configured to deliver power from a remote location relative to the apparatus.

In other embodiments of the apparatus, a tube is provided that has at least one pathway therethrough, and the tube is made from at least one shape-transforming material. Furthermore, the tube is configured for airway passage insertion, which includes configurations for co-navigation through the airway passage.

An embodiment of the apparatus provides that the apparatus is used in imaging via an airway that includes, but is not limited to, at least one of a nasal cavity, an entrance to a visceral tract, a pharynx, a trachea, a larynx, a nares, a mouth, a sinus, an oropharynx, a bronchus, a bronchiole, an alveoli, an entrance to a respiratory tract, a stoma, a ventilator, a tracheostomy and/or a cricothyroidotomy.

A further aspect provides a method of airway imaging. In an embodiment, the method comprises imaging an airway and includes capturing an image adjacent to an airway tube in a proximity to an airway passage. The method further comprises imaging the airway passage using at least one modality to capture images of anatomical structures adjacent to the airway passage and continually imaging over periods of time said airway passage and said anatomical structures adjacent to said airway passage. The method further includes actuating a shape-transformation of an airway tube in response to an image, and at least one shape-transformation of the airway tube. Another embodiment of the method of imaging includes capturing an image from an airway tube that is distal to an airway passage. In other embodiments, the method further includes, capturing a plurality of images from an airway tube placed in an airway during prolonged airway intubation, and for facilitating visualization of the airway and continually monitoring the airway passage during and following a surgical procedure. The method further includes continually monitoring by imaging one or more anatomical structures adjacent to the airway passage including intra-thoracic tissues and/or organs. The method of intubation further includes capturing an image adjacent to an airway tube in proximity to an airway that includes capturing an image using an image-acquisition device configured on a stylet. In an embodiment the method of imaging further includes delivering pharmacologically active chemicals through an airway tube in proximity to an airway. Another embodiment of airway imaging comprises capturing an image adjacent to an airway tube proximate to an airway, which includes capturing the image using an image-acquisition device configured on the airway tube. The method of airway imaging further includes transmitting an image adjacent to an airway tube in proximity to the airway to a visual display, the visual display being located remotely relative to the airway tube. The method of airway imaging also includes transmitting an image adjacent to a stylet in proximity to the airway to a visual display, the visual display being located remotely relative to the airway tube.

An embodiment of the method of imaging further includes inserting a first end of an airway tube; and adjusting a location of the airway tube without removing the airway tube from the airway, and actuating a shape-transformation of an airway tube, in response to an image in at least one shape-transformation of the airway tube that includes guiding the airway tube through the airway passage by actuating at least one shape-transformation of the airway tube. In an embodiment of the method of imaging via an airway, an actuating of the airway tube, in response to the image, at least one shape-transformation of the airway tube includes actuating a shape memory alloy disposed in the airway tube. In yet another embodiment, wherein the actuating a shape-transformation of an airway tube, in response to an image, and at least one shape-transformation of the airway tube includes a steady-state configuration of the airway tube in a fixed direction during an imaging procedure by using one or more force sensors and/or by using one or more devices for adjusting the configuration of an airway tube. In a further embodiment, the actuating a shape-transformation of an airway tube, in response to an image, at least one shape-transformation of an airway tube includes actuating an ex-vivo and/or in vivo shape-transformation by an application of at least one of temperature, electricity, electromagnetic energy, magnetic force, microwave energy, acoustic energy or pressure on the airway tube. Furthermore, actuating a shape-transformation of an airway tube, in response to an image, and at least one shape-transformation of the airway tube includes application of one or more temperature cycles or temperature profiles on the airway tube.

The method of imaging further comprises inserting a stylet into an airway tube. In another embodiment, the method includes inserting a stylet having at least one shape-transforming material. The method further comprises inserting a stylet having one or more sensors into the airway tube, and transmitting information from at least one or more sensors. In other embodiments, the method further comprises inserting a stylet having one or more sensors into the airway tube and transmitting information from at least one of the one or more sensors to a remote location. In an embodiment, the method further comprises parallel imaging of the airway passage concurrently utilizing visual, acoustic, X-ray and/or ultrasound imaging devices that are configured to operate simultaneously in conjunction with each other. In yet another embodiment, a shape-transformation of an airway tube, in response to the image, at least one shape-transformation of the airway tube includes steering the airway tube in a direction coincident with an airway.

In some embodiments, the method of imaging includes capturing an image adjacent to an airway tube proximate to an airway passage and includes displaying the image. Furthermore, the displaying an image includes displaying two-dimensional images. In other embodiments, the displaying includes displaying three-dimensional images. In yet another embodiment, the displaying image includes displaying two-dimensional images and/or three-dimensional images of tissues.

In an embodiment, the method of imaging further includes detecting elemental composition levels in a tissue including detecting concentrations of at least one of calcium, iron or iodine in a tissue.

In addition to the foregoing, other system aspects are described in the claims, drawings, and text forming a part of the present disclosure. Furthermore, various other method and/or system and/or program product aspects are set forth and described in the teachings such as text (e.g., claims and/or detailed description) and/or drawings of the present disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a system-level illustration of an exemplary medical device in which embodiments such as a tube portion and an exemplary stylet carrying an illustrative embodiment of shape-transforming material may be implemented;

FIG. 2 is a schematic of a medical device that includes a tube portion and an exemplary illustrative embodiment of a sensor that includes an exemplary illustrative image-acquisition device;

FIG. 3 is a schematic of a medical device, which includes a tube portion including an exemplary illustration of a sensor that includes an exemplary illustrative data-transmission device;

FIG. 4 is a schematic of a medical device including a tube portion including an exemplary illustration of a sensor that includes an illustrative example of an image-transmission device;

FIG. 5 is a schematic of a medical device including an exemplary illustration of a stylet carrying an exemplary illustration of a sensor that includes an exemplary illustrative data-transmission device;

FIG. 6 is a schematic of a medical device that includes an exemplary illustration of a stylet carrying an exemplary illustration of a sensor that includes an exemplary illustrative data-transmission device illustratively disposed inside the stylet.

FIG. 7 is a schematic of a medical device including an exemplary illustration of a stylet carrying an exemplary illustration of a sensor that includes an exemplary illustration of an image-transmission device;

FIG. 8 is a schematic of a medical device that includes an exemplary illustration of a tube portion carrying an exemplary illustration of a sensor that includes an exemplary illustrative image-acquisition device including an exemplary illustrative signal-receiving-transmitting device;

FIG. 9 is a schematic of a medical device including an exemplary illustration of a tube portion carrying an exemplary illustration of a wireless image display;

FIG. 10 is a schematic of a medical device including an exemplary illustration of a tube portion carrying an exemplary illustration of a sensor that includes an exemplary illustrative data-transmission device and an illustrative optical fiber;

FIG. 11 is a schematic of a medical device including an exemplary illustration of a stylet carrying an exemplary illustration of a sensor that includes illustrative of a data-transmission device disposed outside the stylet;

FIG. 12 is a schematic of a medical device including an exemplary illustration of a tube portion carrying an exemplary illustration of a sensor that includes illustrative of an image-transmission device and an exemplary illustration of an image display;

FIG. 13 is a schematic of a medical device including an exemplary illustration of a tube portion carrying an exemplary illustration of a sensor that includes an image-transmission device coupled to an exemplary illustration of an image display;

FIG. 14 is a schematic of a medical device including an exemplary illustration of a tube portion carrying an exemplary illustration of a sensor that includes an illustrative image-transmission device and an exemplary illustration of a conduit or channel;

FIG. 15 is a schematic of a medical device including an exemplary illustration of a stylet carrying an exemplary illustration of a sensor that includes an exemplary illustrative data-transmission device and an exemplary illustration of a conduit or channel;

FIG. 16 is a schematic of a medical device including an exemplary illustration of a tube portion carrying an exemplary illustration of a stylet;

FIG. 17 is a schematic of a medical device including an exemplary illustration of a tube portion carrying an exemplary illustration of a stylet and exemplary illustration of a visual display proximately located to the tube portion;

FIG. 18 is a schematic of a medical device including an exemplary illustration of a tube portion carrying an exemplary illustration of a stylet and exemplary illustration of a visual display proximately located to the stylet;

FIG. 19 is a schematic of a medical device including an exemplary illustration of a tube portion carrying an exemplary illustration of a stylet and exemplary illustration of a visual display and an exemplary illustration of a power source;

FIG. 20 is a schematic of an apparatus that illustrates exemplary embodiments such as airway intubation structure carrying and an exemplary stylet;

FIG. 21 illustrates an exemplary apparatus which carries an illustrative exemplary airway intubation structure and an exemplary stylet and an exemplary signal generator;

FIG. 22 illustrates an exemplary apparatus which carries an illustrative exemplary airway intubation structure and an exemplary stylet and an exemplary visual display;

FIG. 23 illustrates an exemplary apparatus which carries an illustrative exemplary airway intubation structure and an exemplary stylet and an exemplary visual display illustratively mounted on the stylet;

FIG. 24 illustrates an exemplary apparatus which carries an illustrative exemplary airway intubation structure and an exemplary stylet and an exemplary visual display illustratively mounted on airway intubation structure;

FIG. 25 is a schematic of an apparatus that illustrates exemplary embodiments such as a tube made from an illustrative exemplary shape transforming material located outside the tube and carrying one or more illustrative examples of sensors;

FIG. 26 is a schematic of an apparatus that illustrates exemplary embodiments such as a tube made from an illustrative exemplary shape transforming material located outside and inside the tube distributed in a non-contiguous manner and carrying one or more illustrative examples of sensors;

FIG. 27 is a schematic of an apparatus that illustrates exemplary embodiments such as a tube made from an illustrative exemplary shape transforming material located outside and inside the tube distributed in a contiguous manner and carrying one or more illustrative examples of sensors inside and outside the tube;

FIG. 28 is a schematic of an apparatus that illustrates exemplary embodiments such as a tube made from an illustrative exemplary shape transforming material and carrying one or more illustrative examples of sensors and an illustrative example of an image acquisition device;

FIG. 29 is a schematic of an apparatus that illustrates exemplary embodiments such as a tube made from an illustrative exemplary shape transforming material and carrying one or more illustrative examples of sensors and an illustrative example of a data-transmission device;

FIG. 30 schematically illustrates a simplified implementation of an apparatus in a human patient;

FIG. 31 schematically illustrates a simplified implementation of a medical device in a human patient;

FIG. 32 illustrates an exemplary operational flow in which embodiments of methods of airway imaging may be implemented;

FIG. 33 illustrates an exemplary operational flow in which embodiments of methods of actuating shape transformation, capturing and image, transmitting an image, delivering a drug, inserting a stylet and detecting a chemical may be implemented;

FIG. 34 illustrates embodiments of an exemplary operational flow for capturing an image;

FIG. 35 illustrates embodiments of an exemplary operational flow for actuating a shape transformation;

FIG. 36 illustrates embodiments of an exemplary operational flow for inserting a stylet;

FIG. 37 illustrates embodiments of an exemplary operational flow for displaying an image;

FIG. 38 illustrates embodiments of an exemplary operational flow for detecting elemental composition levels.

### DETAILED DESCRIPTION

In the following detailed description, reference is made to the accompanying drawings, which form a part hereof. In the drawings, similar symbols typically identify similar components, unless content dictates otherwise. The illustrative embodiments described in the detailed description, drawings, and claims are not meant to be limiting. Other embodiments may be utilized, and other changes may be made, without departing from the spirit or scope of the subject matter presented here.

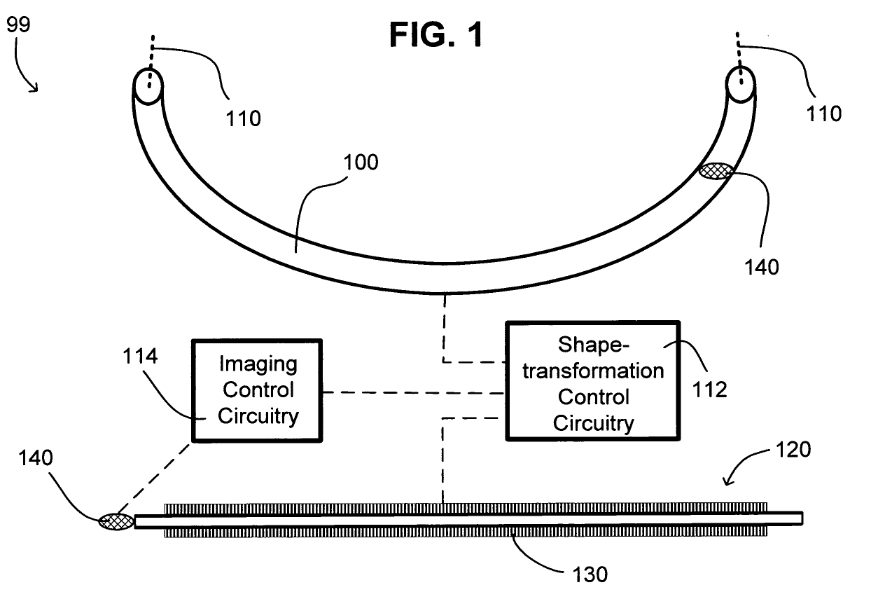

The following disclosure is drawn to a medical device. FIG. 1 is a schematic system-level illustration of one embodiment of a medical device 99, which comprises a tube portion 100 shaped and sized for airway passage insertion. The tube includes a pathway 110 therethrough. In another embodiment, the medical device further comprises a stylet 120 of at least one shape-transforming material 130, configured for insertion in the pathway of the tube portion. In some embodiments the stylet carries at least one sensor 140. In other embodiments, the tube portion may also carry a sensor 140. The sensor may be located inside and/or outside and/or at one or the other end of the tube portion and/or the stylet. In addition, FIG. 1 illustrates an embodiment of the medical device that includes, but is not limited to, electrical circuitry 112 that controls shape transformation of the stylet 120 and/or the tube portion 100. Furthermore, FIG. 1 illustrates optional electrical circuitry that controls imaging 114. The broken lines in FIG. 1 are illustrative of communication systems between one or more of the tube portions, the stylet and/or the sensor, and one or more of the imaging circuitry and/or the shape transformation circuitry. In some embodiments, the communication systems include, but are not limited to, any appropriate form of communication media such as an optical fiber, a waveguide, a nanotube, a metal wire and/or a nonmetallic wire. In one or more of the various embodiments, related systems include but are not limited to, electrical circuitry and/or programming for effecting communications. The circuitry and/or programming can be virtually any combination of hardware, software, and/or firmware configured to effect the communication depending upon the design choices of the system designer.

As used herein, the terms “tube” or “tube portion” or “intubation structure” or “airway tube” include, but are not limited to, an entire endotracheal tube or parts thereof and/or similar intubation devices, that are used in any medical or surgical care including endoscopy, anesthesia, intensive care and emergency medicine for airway management, imaging, intubation, mechanical ventilation and/or in suction ports. The size ranges of a tube portion referred to herein may be of any length or width and is not limited by body size or body mass of a patient. In some embodiments this includes tubes selected based on body size and/or body mass.

In an embodiment, the terms “body” or “patient” refer to a human or any animal including domestic, marine, research, zoo, farm animals, fowl and sports animals, or pet animals, such as dogs, cats, cattle, horses, sheep, pigs, goats, rabbits, chickens, birds, fish, amphibian and reptile.

In an embodiment, the term “stylet” refers to, but is not limited to, any of the medical instrument means that is inserted either partly or completely into a tube portion or is capable of penetrating or piercing a tube portion. The stylet may be used for any medical or surgical care including imaging, endoscopy, anesthesia, intensive care and emergency medicine for airway management, intubation, mechanical and/or a suction port.

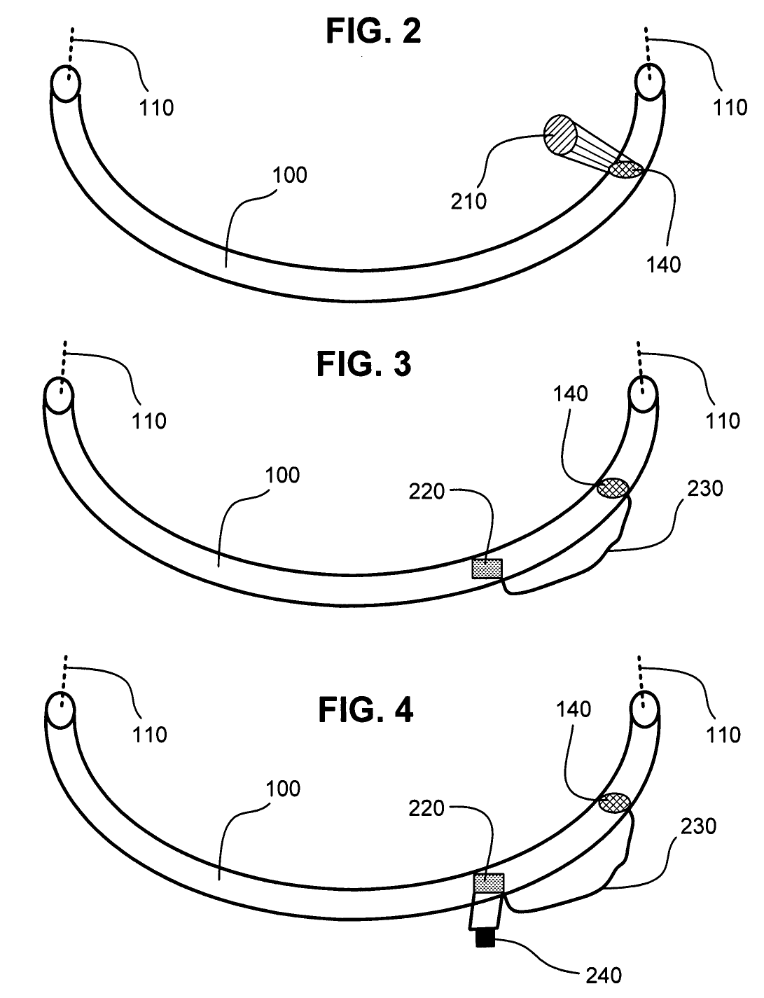

As illustrated in FIG. 2, in one embodiment, a medical device provides for a tube portion 100 that includes at least one sensor 140, which may include an exemplary image-acquisition device 210. In some embodiments, the image-acquisition device may include, but is not limited to, at least one of the following types of devices: cameras, charge coupled devices, scanning cameras, cellular-technology based image-acquisition devices, digital or analogue type devices, still or motion picture cameras, telescopic cameras, stereo cameras, infrared or optical or laser or ultrasound-based image acquisition devices, speed cameras, instant cameras, folding cameras, disposable cameras, hand-held or fixed cameras, video cameras, multichannel or single channel computer-based cameras, prosumer cameras, reflex single or multi-lens cameras, disc cameras, electronic imaging cameras, night vision cameras and/or miniature image acquisition devices incorporated into PDAs, watches, quantum dots, nanotubes/sheets and cell phones. In other embodiments, image-acquisition devices may include at least one of an X-ray receiver, an acoustic energy receiver, an electromagnetic energy receiver and/or an imaging device.

FIG. 3 illustrates an exemplary medical device that includes at least one data-transmission device 220 disposed inside a tube portion 100. In other embodiments, the data-transmission device may be disposed outside the tube portion. The data-transmission device is configured to operatively communicate via a medium 230 with at least one sensor 140. The communication medium may include, but is not limited to, any appropriate form of communication media such as optical fiber, waveguide, nanotube, metal wire and/or nonmetallic wire, or other appropriate media for communicating signals or information.

As illustrated in FIG. 4, in one embodiment at least one data-transmission device 220 includes an image-transmission device 240, which may be disposed either in (or outside) a tube portion 100. In a further embodiment, the data-transmission device is configured to operatively communicate with at least one sensor 140. The communication may be via communication medium 230 that includes, but is not limited to, any appropriate form of communication media such as one or more optical fibers, waveguides, nanotube, metal wire and/or nonmetallic wire, or other appropriate media for communicating signals or information.

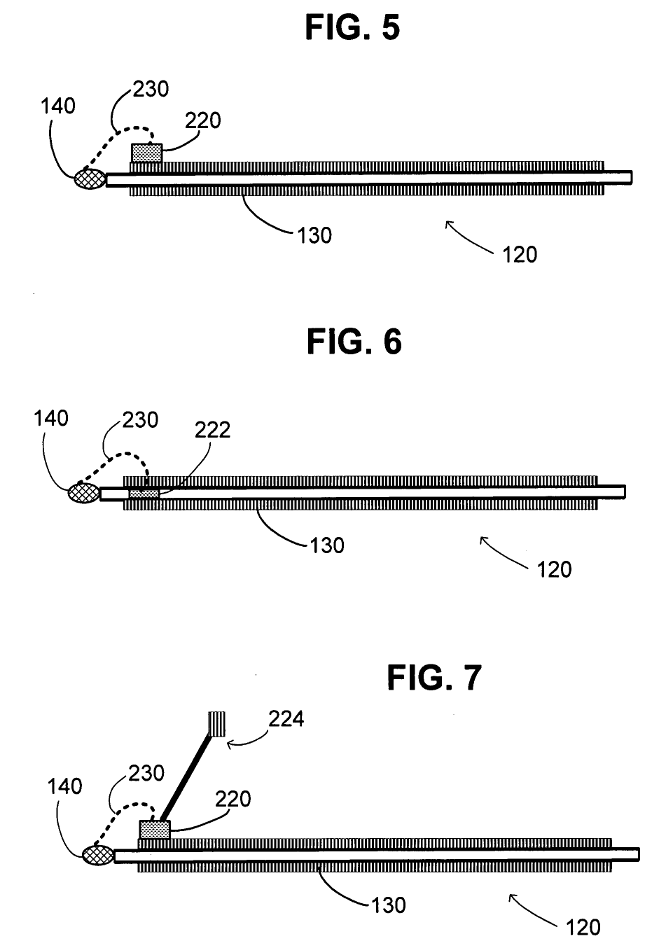

FIG. 5 schematically illustrates an exemplary stylet 120 that includes but is not limited to, at least one data-transmission device 220 disposed outside the stylet. The at least one data-transmission device is configured to operatively communicate with the at least one sensor 140. The communication may be via communication media 230 that include, but are not limited to, any appropriate form of communication media such as an optical fiber, a waveguide, a nanotube, a metal wire and/or a nonmetallic wire. As depicted in FIG. 6, in other embodiments, the data-transmission device 222 may be disposed inside the stylet.

As illustrated in FIG. 7, in alternative embodiments of a stylet 120, at least one data-transmission device 220 includes an image-transmission device 224. Examples of image and data transmission devices include, but are not limited to, single or multichannel transmitting devices, robotic and/or non-robotic transmission devices, miniature transmitters and receivers (E.g., see U.S. Pat. Nos. 5,305,116, 6,166,729, 7,023,573 and 7,110,860).

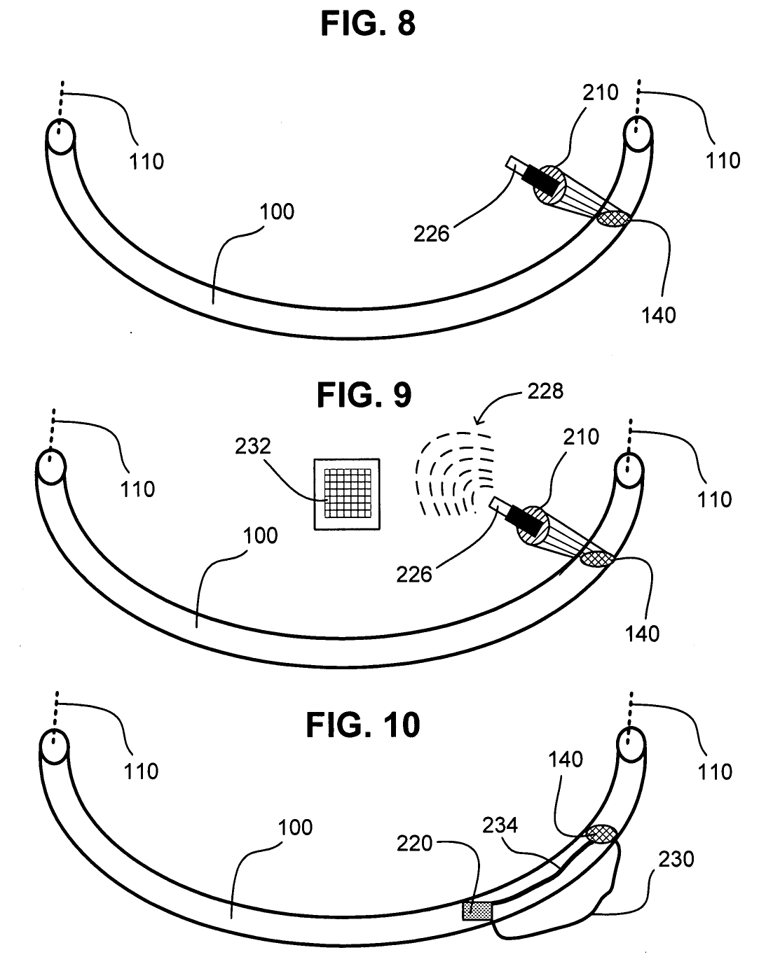

Turning now to FIG. 8, in some embodiments, a medical device includes a tube portion 100 and image-acquisition device 210 that includes at least one of a signal-receiving-transmitting device 226. Alternative embodiments of the image-acquisition device include, but are not limited to, a camera, a charged coupled device, a scanner, an X-ray receiver, an acoustic energy receiver, an electromagnetic energy receiver and/or an imaging device.

In FIG. 9 a further illustrative example of a tube portion 100 is shown. The tube portion comprises a signal-receiving-transmitting device 226 that is wirelessly 228 coupled to at least one visual display 232. Wireless coupling includes, but is not limited to, communication operations using electronic signals, electrical signals, wave propagation signals, acoustic, electromagnetic and/or photonic radiation. Such communication operations may be short range and/or long range. Typical systems utilizing wireless operation include radio transmitters and receivers, remote controls, computer networks, network terminals, etc.), which use some form of energy (e.g., radio frequency (RF), infrared light, laser light, visible light, acoustic energy, etc.) Wireless systems of communication may or may not be “cordless or mobile” and do not preclude hardwiring of systems, and digital or analog systems.

FIG. 10 illustrates an embodiment of a tube portion 100 having at least one sensor 140 and at least one data-transmission device 220. In an embodiment, there is illustrated an operational communication between the at least one sensor and the at least one data-transmission device. The operational communication includes at least one of a communication medium 234. For instance, the communication medium may include any appropriate signal-carrying path or device such as an optical fiber, a waveguide, a nanotube, a metal wire and/or a nonmetallic wire or other appropriate media for communicating signals or information.

FIG. 11 illustrates an embodiment of an exemplary stylet 120 that includes at least one sensor 140 that is in operational communication with a data-transmission device 220. The operational communication includes at least one communication medium 230. The communication medium includes any appropriate signal-carrying path or device such as an optical fiber, a waveguide, a nanotube, a metal wire and/or a nonmetallic wire.

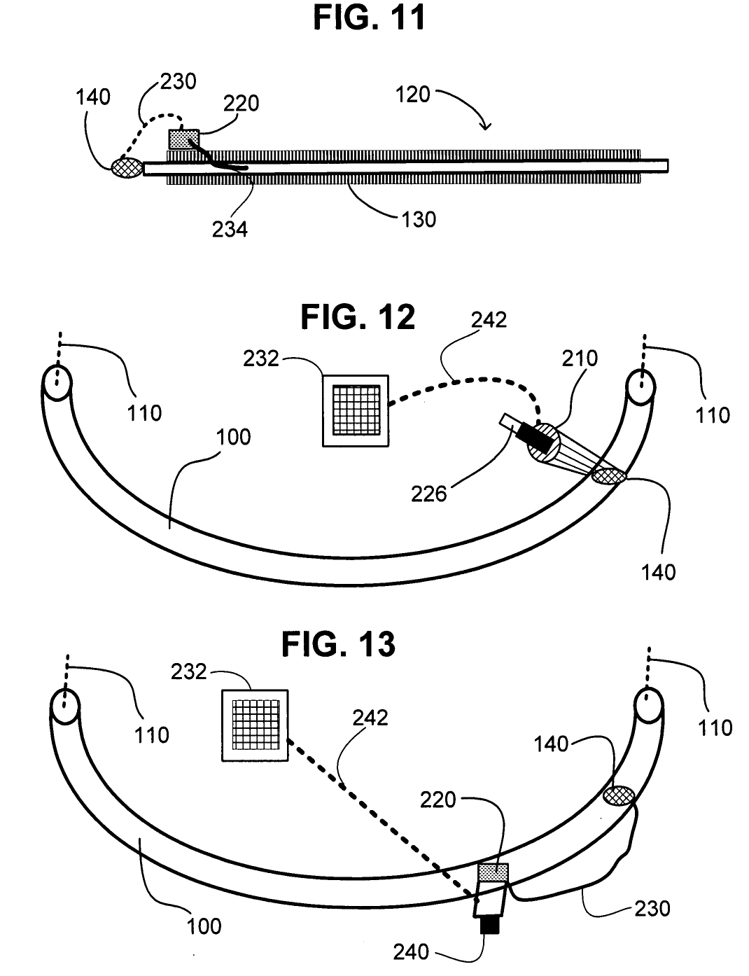

FIG. 12 illustrates an exemplary embodiment of a tube portion 100 of a medical device in which, at least one signal-receiving-transmitting device 226 is configured to operably communicate through a medium 242 with at least one visual display 232. The communication medium includes any appropriate signal-carrying path or device such as an optical fiber, a waveguide, a nanotube, a metal wire and/or a nonmetallic wire. Visual displays include, but are not limited to, cathode ray tubes, television screens, liquid crystal displays, surface-conduction electron-emitter displays, vector displays, video projectors, computer monitors, computer terminals, TTL monitors, digital and/or analogue monitors, miniature displays, single or multi channel monitors, virtual displays, color or black and white displays, screenless computing, multimedia displays and/or multihead displays.

FIG. 13 schematically illustrates an embodiment of a tube portion that includes a visual display 232 that is coupled to a communication medium 242 that communicates with an image-transmission device 240. In an alternative embodiment, the visual display may be in communication with a data-transmission device 220. In another embodiment, a sensor 140 in the tube portion may be placed in communication with a data transmission device 220 via a communication medium 230. The communication medium includes any appropriate signal-carrying path or device such as an optical fiber, a waveguide, a nanotube, a metal wire and/or a nonmetallic wire.

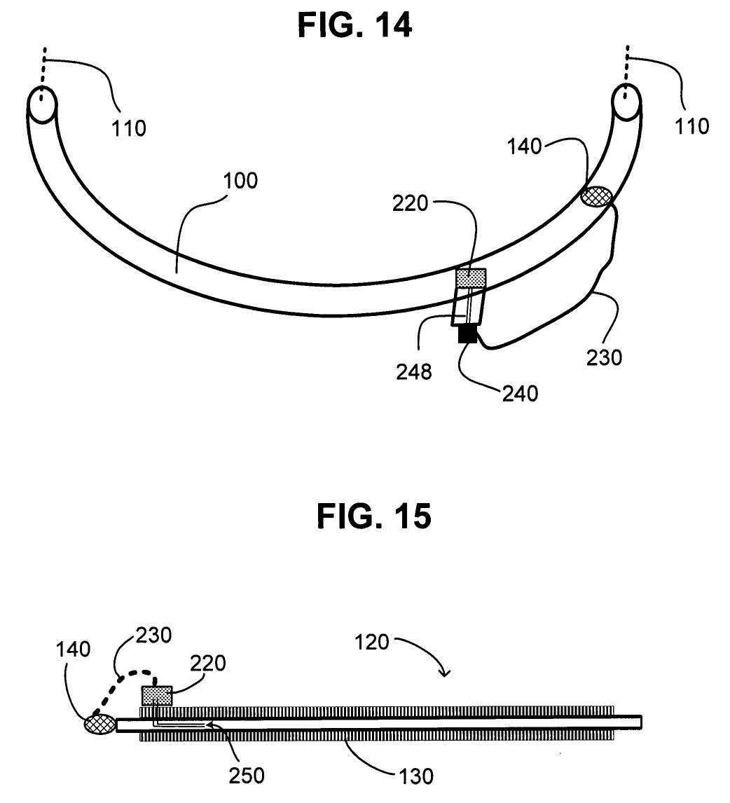

FIG. 14 schematically illustrates an embodiment of a tube portion 100 that includes an image-transmission device 240. In some embodiments, the image-transmission device includes a conduit and/or channel 248. In alternative embodiments, the channels or conduits may be used for parallel imaging using a variety of modalities such as optical cameras, X-ray emitters and/or acoustic devices. In one embodiment, the image-transmission device 240 may be operationally coupled to a sensor 140 via a communication medium 230. The communication medium includes any appropriate signal-carrying path or device such as an optical fiber, a waveguide, a nanotube, a metal wire and/or a nonmetallic wire.

FIG. 15 schematically illustrates an embodiment of a stylet 120, and a data-transmission device 220 that includes a conduit and/or channel 250. In alternative embodiments the channels or conduits may be used for parallel imaging using a variety of modalities such as optical cameras, X-ray emitters and/or acoustic devices. In one embodiment, the data-transmission device 220 may be operationally coupled to a sensor 140 via a communication medium 230. The communication medium includes any appropriate signal-carrying path or device such as an optical fiber, a waveguide, a nanotube, a metal wire and/or a nonmetallic wire.

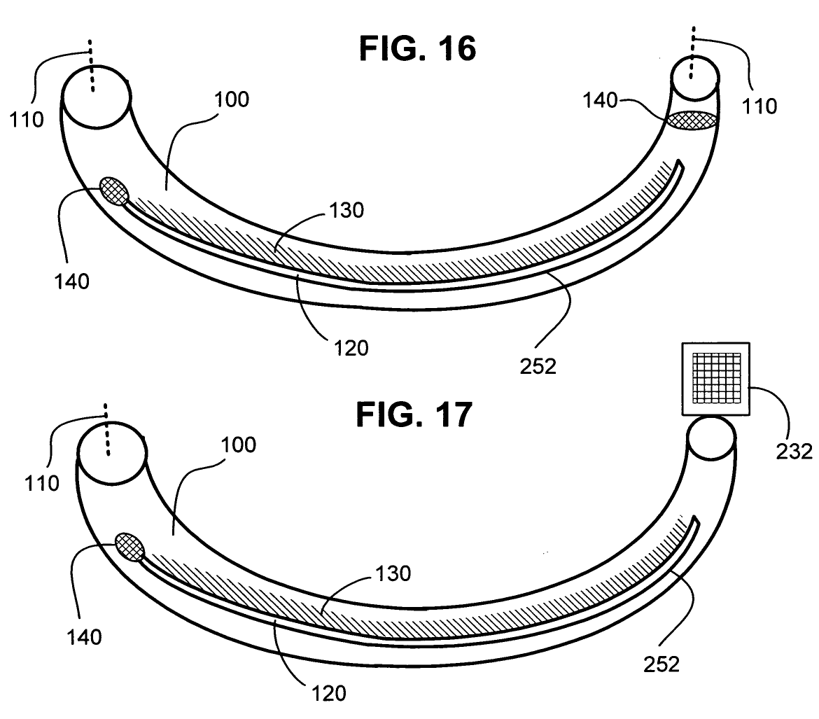

FIG. 16 schematically illustrates an embodiment of a medical device having a stylet 120 that is configured and/or shaped according to an appropriate pattern or curvature 252 to fit into a tube portion 100. Configuration of the stylet includes, but is not limited to, shape-transformation mediated by shape-transforming material 130 such as a shape memory alloy, Nitinol™, electro-active polymer and/or a mechanically reconfigurable material. In one embodiment, the shape-transformation is actuated by at least one of a voltage profile, a temperature profile, a magnetic force field, a steering force, a pressure profile, an electrical current and/or a solid-state phase change.

FIG. 17 illustrates an embodiment of a stylet 120 and a tube portion 100 having at least one visual display 232 that is located in a proximity to the tube portion. In other embodiments, at least one visual display may be attached as shown, to the tube portion itself or may be wired to a part of the tube portion.

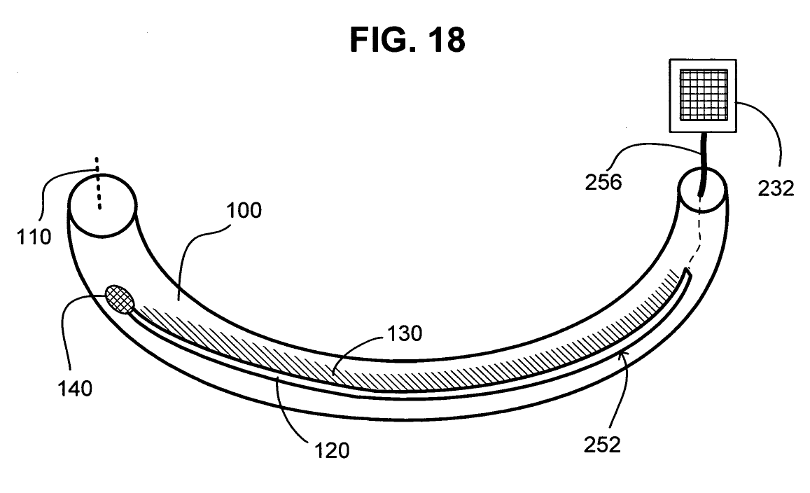

FIG. 18 illustrates an embodiment of a stylet 120 and a tube portion 100 having at least one visual display 232 is located in a physical proximity to the stylet. In some embodiments, at least one visual display 232 may be operably coupled 256 to a part of the stylet. The coupling system may include, but is not limited to, any appropriate hardwiring and/or cordless type of communication device. In an embodiment, the coupling may include physical attachment to the stylet itself.

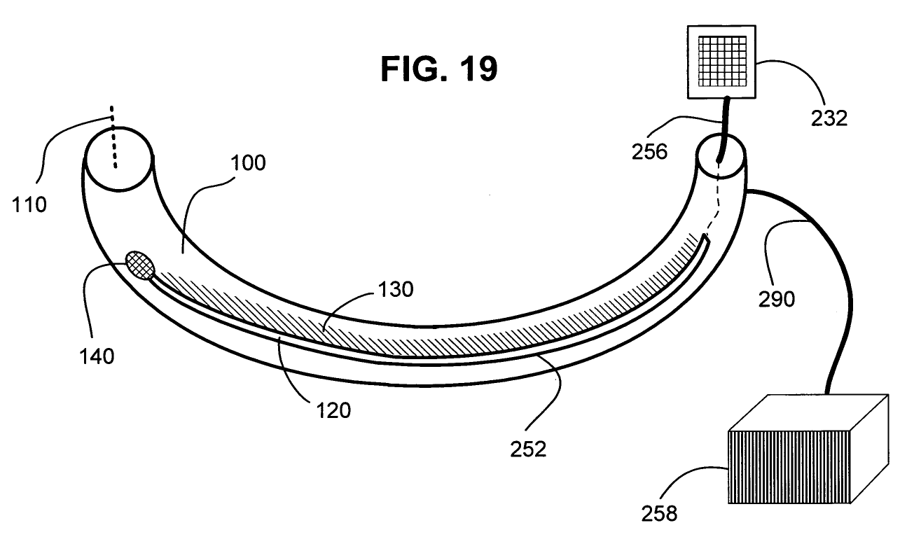

FIG. 19 illustrates an embodiment of a medical device comprising at least one power source 258. In alternative embodiments, the power source is mounted on the medical device and/or otherwise operably coupled 290 to the tube portion 100 of the medical device. In some embodiments, at least one power source is configured to deliver power from a remote location relative to the medical device.

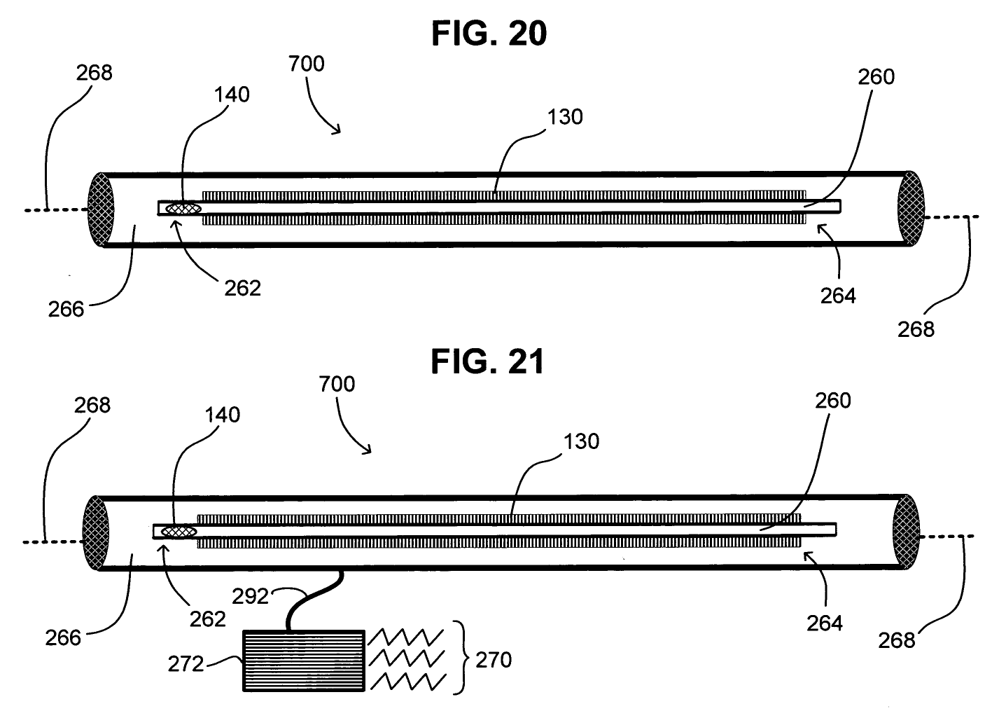

FIG. 20 schematically illustrates an embodiment of an apparatus 700 comprising at least one stylet 260 having a first end 262 and a second end 264, and at least a portion of the stylet includes a shape-transforming material 130. The stylet is configured for insertion through an airway intubation structure 266 having at least one pathway 268 therethrough. In an other embodiments, the stylet carries at least one sensor 140 in a proximity to the first end 262. In a further embodiment, of the apparatus, at least one sensor is located at the second end of the stylet. Other embodiments may include, but are not limited to, sensors located at multiple places either in the stylet and/or in the airway intubation structure.

FIG. 21 illustrates an alternative embodiment of an apparatus 700 comprising at least one airway intubation structure 266 is operably coupled 292 to at least one audio signal 270 generator 272. In alternative embodiments, an audio signal generator may be operably coupled to at least one sensor, a data-transmission device, an image transmission device and/or a stylet. In some embodiments, the coupling of an audio signal generator may be through a wireless system or a cordless system and/or through a hardwired system.

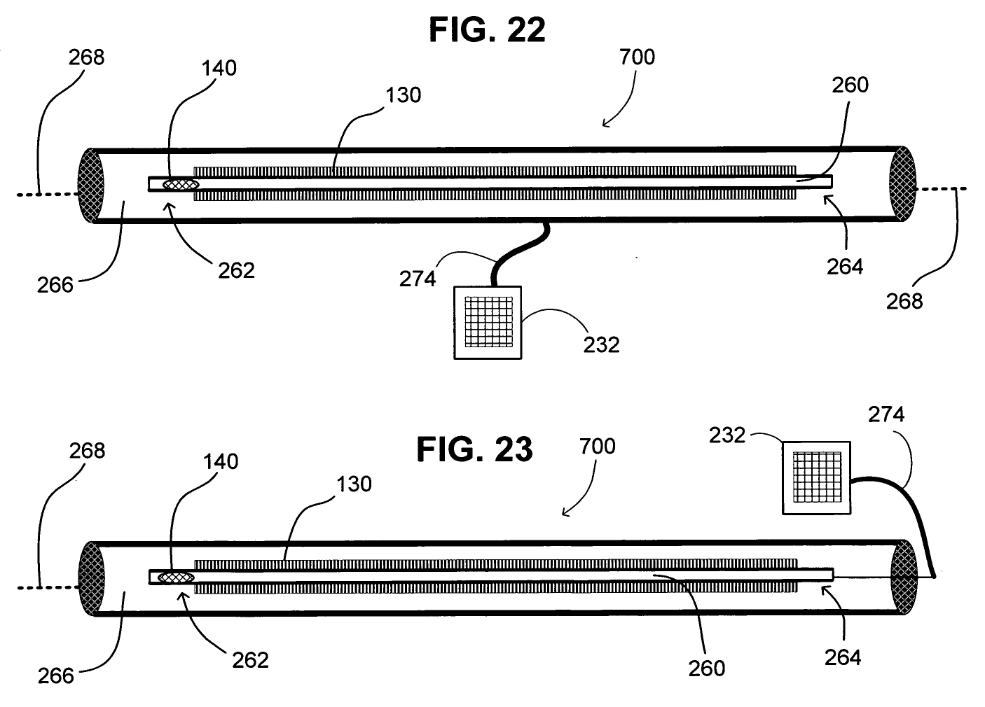

In FIG. 22, there is illustrated a further embodiment of an apparatus 700 comprising airway intubation structure 266 that is operably coupled 274 to at least one visual display 232. In alternative embodiments, at least one visual display may be operably coupled to at least one sensor, a data-transmission device, an image transmission device, an audio signal generator and/or a stylet. In some embodiments, the coupling of a visual display and/or an audio signal generator may be through a wireless system and/or through hardwired system.

Turning to FIG. 23, an alternative embodiment of an apparatus 700 is illustrated which includes at least one visual display that is operably connected via a medium 274 and/or is mounted on a stylet 260.

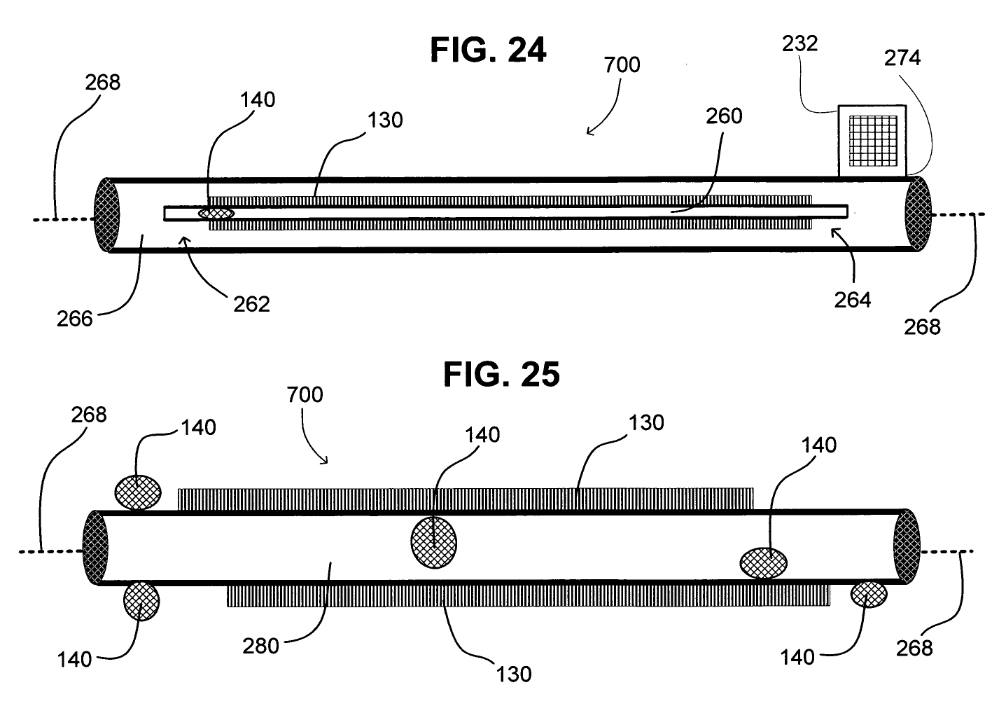

FIG. 24 illustrates yet another embodiment of an apparatus 700 is illustrated in which, at least one visual display is operably coupled 274 and/or is mounted on an airway intubation structure 266.

A further embodiment is disclosed in FIG. 25, which illustrates an exemplary apparatus 700 that includes a tube 280 having at least one pathway 268 therethrough, the tube being made from at least one shape-transforming material 130, and configured for airway passage insertion. In a further embodiment, the tube carries multiple sensors 140 disposed in various locations within and/or on the outside surface of the tube. In yet another embodiment, the tube may carry many sensors located at various places as shown in FIG. 25.

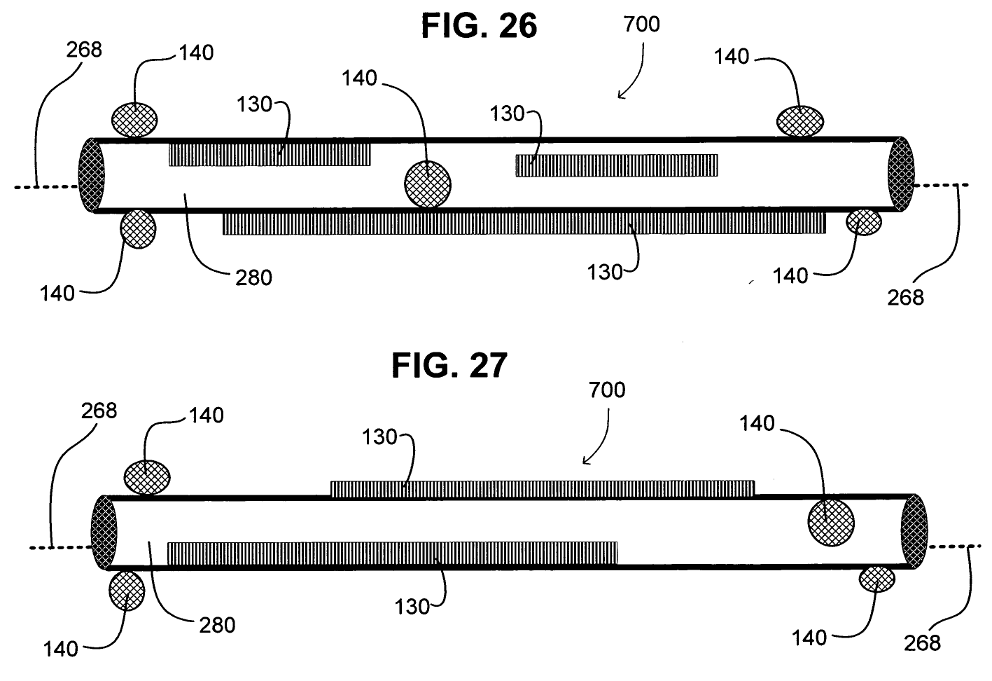

FIG. 26 illustrates yet another embodiment of an apparatus 700 that includes at least one shape-transforming material 130 that may be distributed either discontiguously inside and/or outside of the tube 280.

FIG. 27 illustrates another embodiment of an apparatus 700 that includes at least one shape-transforming material 130 that may be distributed asymmetrically either in and/or outside the tube 280. In some embodiments, the asymmetric distribution of the shape-transforming material may be partially or fully integrated or molded into the tube.

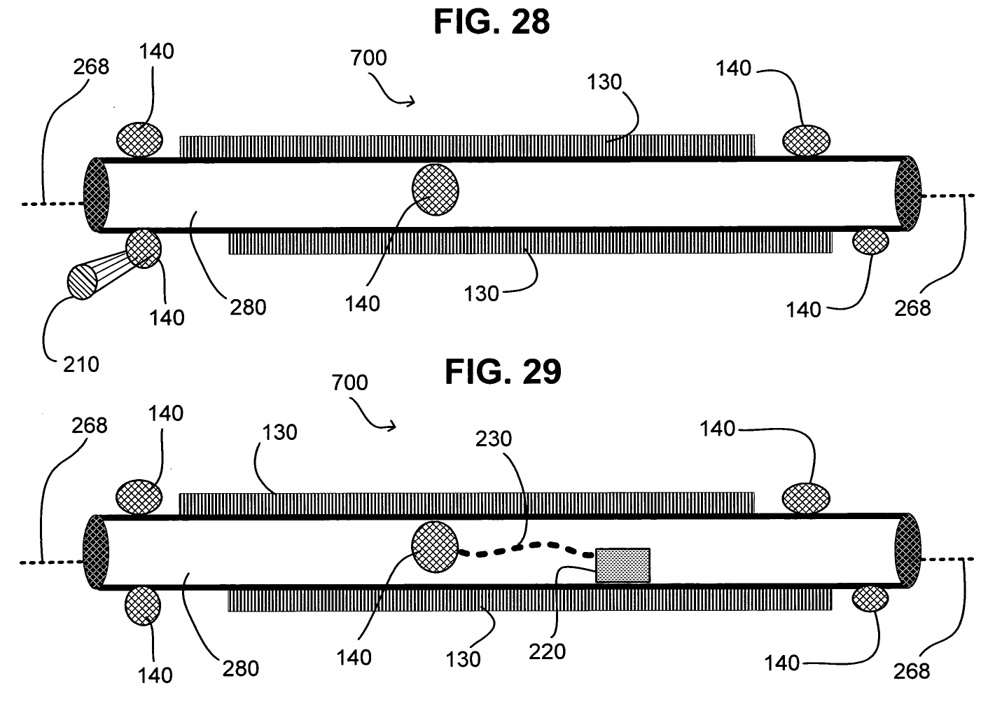

FIG. 28 illustrates a further embodiment of an apparatus 700 that includes at least one sensor 140 that includes an image-acquisition device 210.

FIG. 29 illustrates yet another embodiment of an apparatus 700 comprising at least one data-transmission device 220 disposed inside a tube 280. Furthermore, at least one data-transmission device 220 is configured to be operatively coupled through a medium 230 to at least one sensor 140. In another embodiment of the apparatus at least one data-transmission device includes an image-transmission device (not shown).

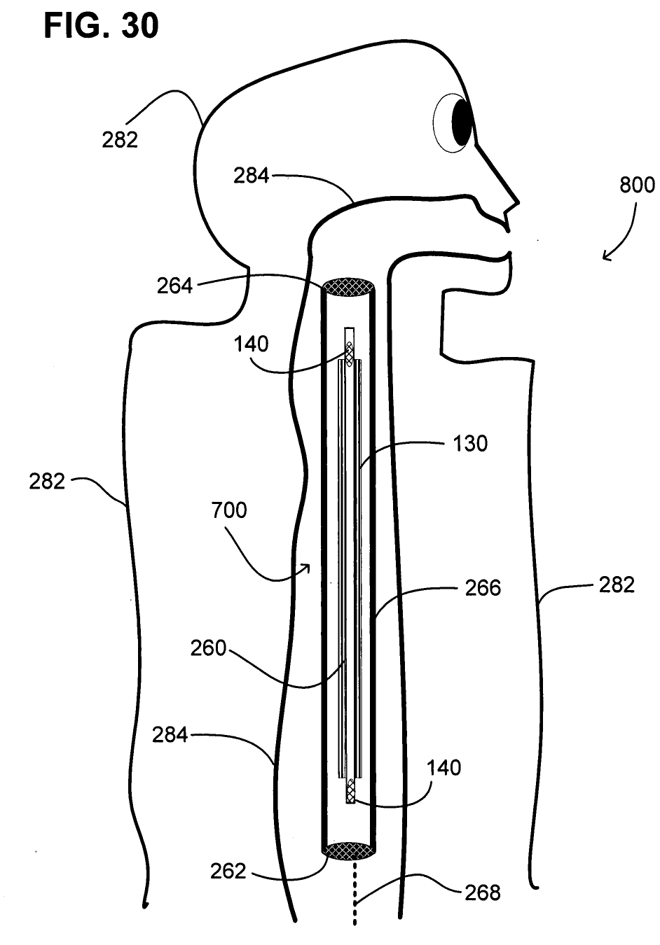

FIG. 30 schematically illustrates a simplified implementation 800 of an apparatus 700 comprising an airway intubation structure 266 in a human patient 282. In some embodiments, the structure is inserted in an airway 284. The structure comprises a tube having at least one pathway 268 therethrough comprising at least one stylet 260 having a first end 262 and a second end 264, and at least a portion of the stylet includes a shape-transforming material 130. In one embodiment, as shown in FIG. 30 the stylet carries multiple sensors 140 at each end. In other embodiments, the sensors may be located at various places either inside and/or outside the stylet (not shown).

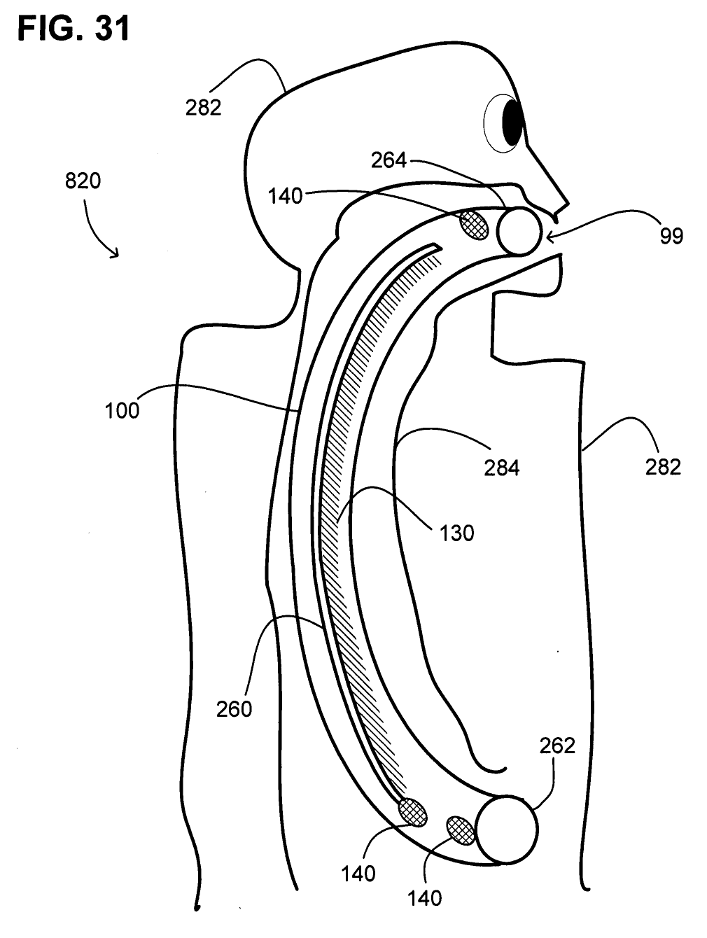

FIG. 31 schematically illustrates a simplified implementation 820 of a medical device 99 comprising a tube portion 100 in a human patient 282. In some embodiments, the medical device is inserted in an airway 284 comprises a tube portion 266 having at least one pathway 268 therethrough comprising at least one stylet 260 having a first end 262 and a second end 264, and at least a portion of the stylet includes a shape-transforming material 130. In one embodiment, as shown in FIG. 31, the tube portion and the stylet carry multiple sensors 140. In other embodiments, sensors may be located at various places either inside and/or outside the stylet (not shown).

Although an illustrative a human patient is shown FIGS. 30 and 31, those skilled in the art will appreciate that humans may be only representative patients. Thus other patients may be envisaged by those skilled in the art. Other patients include, but are not limited to, an animal, a robotic simulator of a human or animal (e.g., computational entity), and/or substantially any combination thereof (e.g., a human or an animal patient may be assisted by one or more robotic agents). In addition, human patient, as set forth herein, although shown as a single entity may in fact be composed of two or more entities.

In yet another embodiment, the tube portion and/or the stylet may be actuated to undergo a shape transformation to facilitate insertion of the tube portion and/or the stylet into the body lumen of the patient. Insertion into a body lumen may include, but is not limited to, anatomical structures such as nasal cavity, entrance to a visceral tract, pharynx, trachea, larynx, nares, mouth, sinus, an oropharynx, bronchi, bronchioles, alveoli, entrance to a respiratory tract, stoma, ventilator, tracheostomy and/or cricothyroidotomy.

In other embodiments of the medical device, an apparatus comprising more than one stylet comprising multiple channels or conduits configured for insertion of different imaging modalities may be inserted into a body lumen of a patient in need of treatment.

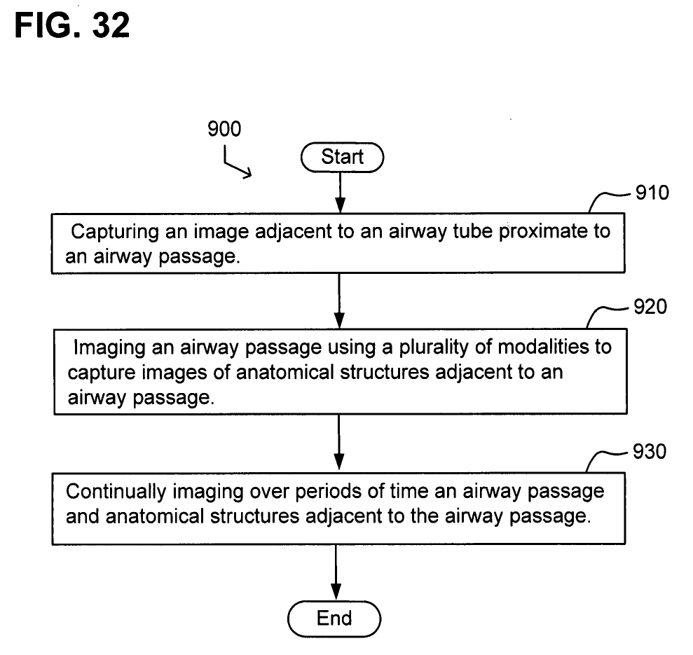

FIG. 32 shows an exemplary operational flow 900 of a method of imaging a patient. In an embodiment the method includes capturing an image adjacent to an airway tube proximate to an airway passage 910. In a further embodiment the method includes imaging an airway passage using a plurality of modalities to capture images of anatomical structures adjacent to an airway passage 920. In yet another embodiment the method includes continually imaging over periods of time an airway passage and anatomical structures adjacent to the airway passage 930.

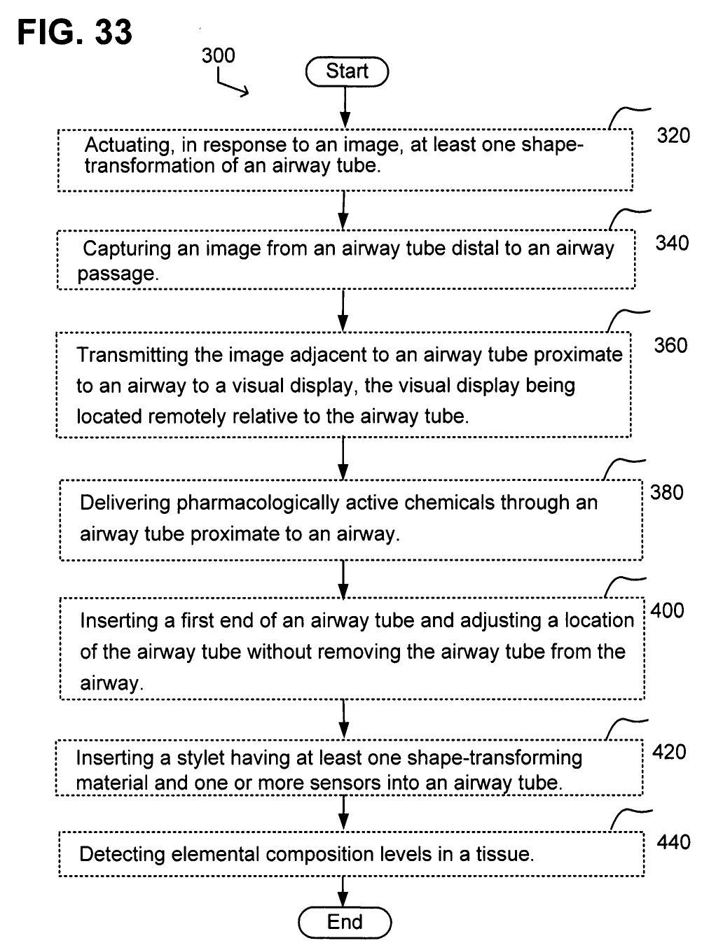

A further embodiment includes a method of imaging a patient. FIG. 33 illustrates embodiments of an exemplary operational flow 300 for capturing an image. An embodiment of the exemplary operational flow includes: (1) actuating, in response to an image, at least one shape-transformation of an airway tube 320; (2) capturing an image from an airway tube distal to an airway passage 340; (3) transmitting the image adjacent to an airway tube proximate to an airway to a visual display, the visual display being located remotely relative to the airway tube 360; (4) delivering pharmacologically active chemicals through an airway tube proximate to an airway 380; (5) inserting a first end of an airway tube and adjusting a location of the airway tube without removing the airway tube from the airway 400; (6) inserting a stylet having at least one shape-transforming material and one or more sensors into an airway tube 420; and (7) detecting elemental composition levels in a tissue 440.

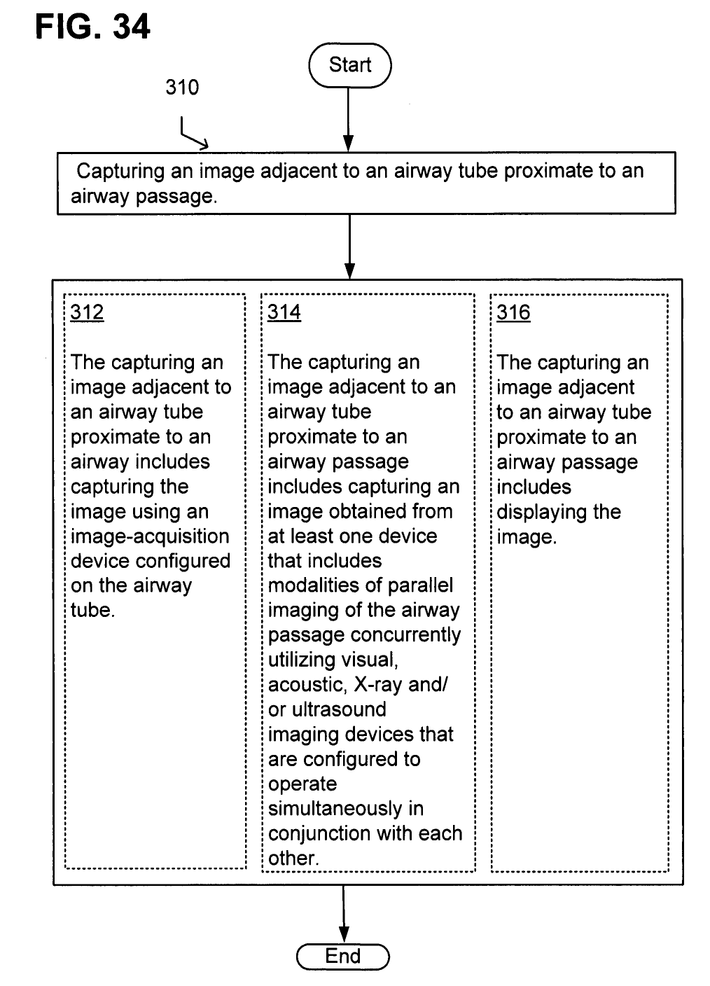

FIG. 34 illustrates embodiments of an exemplary operational flow for capturing an image adjacent to an airway tube proximate to an airway passage 310. The operation includes, but is not limited to: (1) capturing an image adjacent to an airway tube proximate to an airway includes capturing the image using an image-acquisition device configured on the airway tube 312; capturing an image adjacent to an airway tube proximate to an airway passage includes capturing an image obtained from at least one device that includes modalities of parallel imaging of the airway passage concurrently utilizing visual, acoustic, X-ray and/or ultrasound imaging devices that are configured to operate simultaneously in conjunction with each other 314; and (3) capturing an image adjacent to an airway tube proximate to an airway passage includes displaying the image 316.

In yet another embodiment, capturing an image includes temporal and spatial variations in imaging adjacent to an airway intubation structure. Temporal variations in imaging include both short term and long term airway imaging. Short term imaging includes, but is not limited to, capturing images, for example, to facilitate the insertion and/or navigation of endotracheal tubes and/or stylets into their respective proper places during intubation. This typically involves short term visualization of endotracheal and associated anatomical structures. Long term imaging includes continually capturing images for prolonged periods of time for monitoring of the patient's airway, which may include imaging the airway over a period of days, weeks, months and/or years. Long term visualization of a patient's airway includes imaging during mechanical ventilation of the airway during surgery and/or during intensive care or critical care.

Other embodiments for capturing an image include spatial variations in the capturing of images. One embodiment of the spatial variation includes capturing images of structures adjacent to the patient's airway. These adjacent structures include, but are not limited to, intra-thoracic tissues (chest, lungs, heart, esophagus, etc.) after the airway intubation structure has been properly placed.

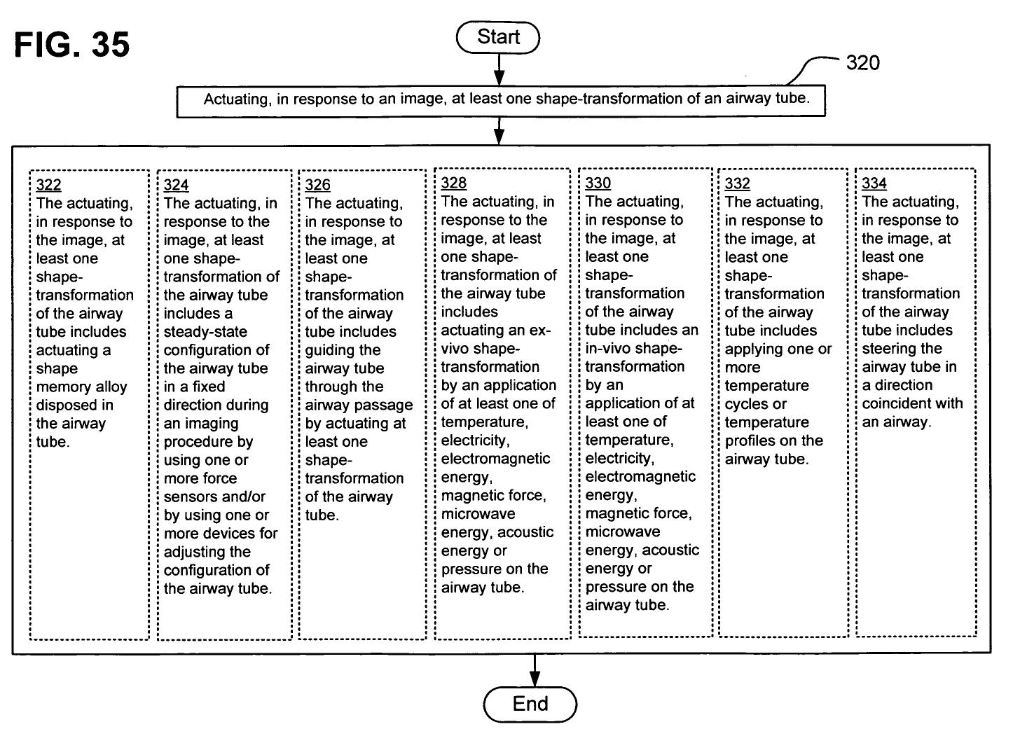

FIG. 35 illustrates embodiments of an exemplary operational flow for actuating, in response to an image, at least one shape-transformation of an airway tube 320. In alternative embodiments the actuating includes, but is not limited to: (1) actuating, in response to the image, at least one shape-transformation of the airway tube includes actuating a shape memory alloy disposed in the airway tube 322; (2) actuating, in response to the image, at least one shape-transformation of the airway tube includes a steady-state configuration of the airway tube in a fixed direction during an imaging procedure by using one or more force sensors and/or by using one or more devices for adjusting the configuration of the airway tube 324; (3) actuating, in response to the image, at least one shape-transformation of the airway tube includes guiding the airway tube through the airway passage by actuating at least one shape-transformation of the airway tube 326; (4) actuating, in response to the image, at least one shape-transformation of the airway tube includes actuating an ex-vivo shape-transformation by an application of at least one of temperature, electricity, electromagnetic energy, magnetic force, microwave energy, acoustic energy or pressure on the airway tube 328; (5) actuating, in response to the image, at least one shape-transformation of the airway tube includes an in-vivo shape-transformation by an application of at least one of temperature, electricity, electromagnetic energy, magnetic force, microwave energy, acoustic energy or pressure on the airway tube 330; (6) actuating, in response to the image, at least one shape-transformation of the airway tube includes applying one or more temperature cycles or temperature profiles on the airway tube 332; (7) actuating, in response to the image, at least one shape-transformation of the airway tube includes steering the airway tube in a direction coincident with an airway 334.

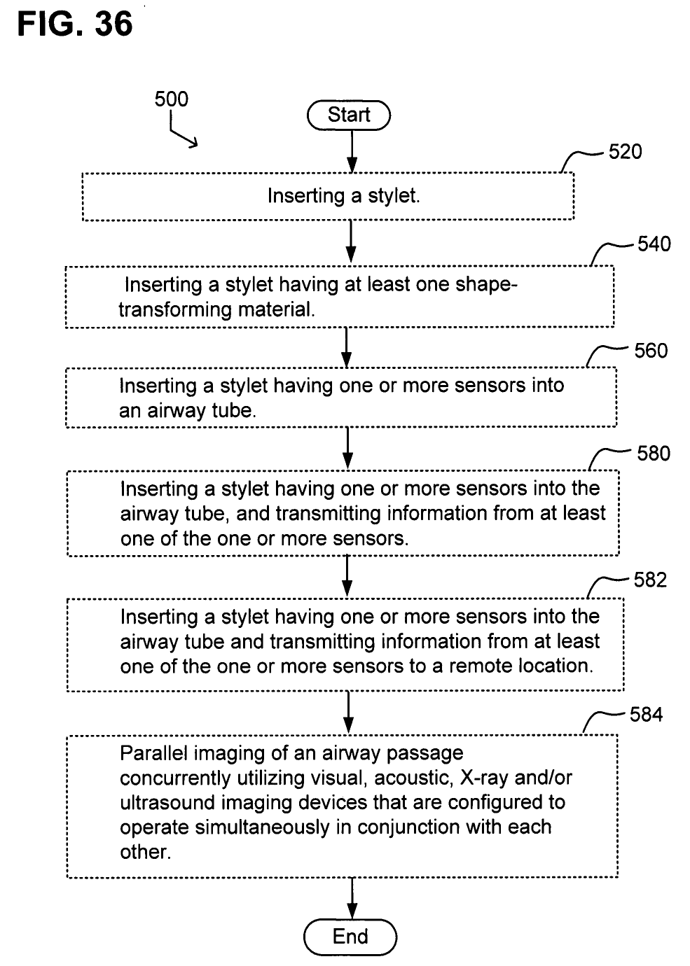

FIG. 36 illustrates embodiments of an exemplary operational flow 500 for inserting a stylet 520. In various embodiments this operational flow includes: (1) inserting a stylet having at least one shape-transforming material 540: (2) inserting a stylet having one or more sensors into an airway tube 560; (3) inserting a stylet having one or more sensors into the airway tube, and transmitting information from at least one of the one or more sensors 580; (4) inserting a stylet having one or more sensors into the airway tube and transmitting information from at least one of the one or more sensors to a remote location 582; (5) parallel imaging of an airway passage concurrently utilizing visual, acoustic, X-ray and/or ultrasound imaging devices that are configured to operate simultaneously in conjunction with each other.

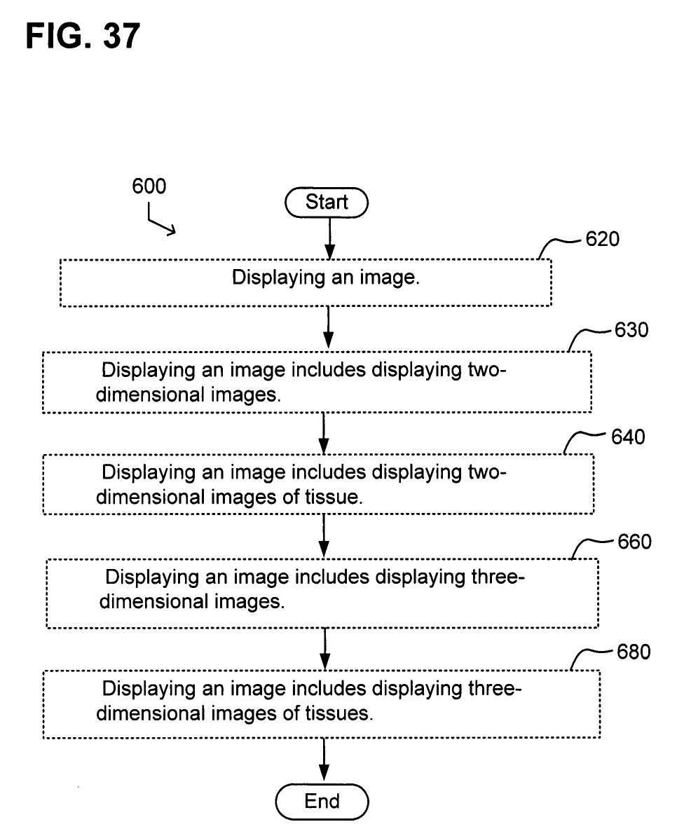

FIG. 37 illustrates embodiments of an exemplary operational flow 600 for displaying an image 620. In another embodiment displaying an image includes at least one the following: (1) displaying the image includes displaying two-dimensional images 630; (2) displaying the image includes displaying two-dimensional images of tissue 640; (3) displaying the image includes displaying three-dimensional images 660; (4) displaying the image includes displaying three-dimensional images of tissues 680.

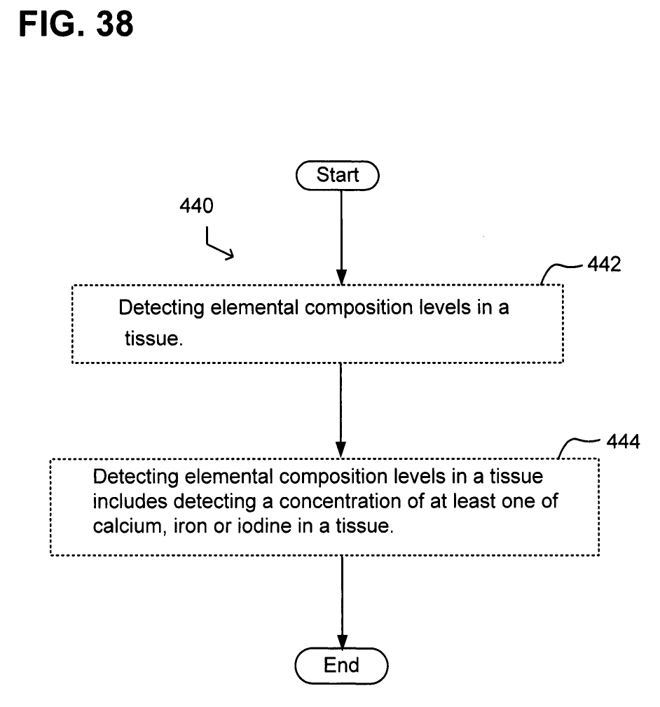

FIG. 38 illustrates embodiments of an exemplary operational flow 440 for detecting elemental composition levels in a tissue 442. In an alternative embodiment detecting elemental composition levels in a tissue includes detecting a concentration of at least one of calcium, iron or iodine in a tissue 444.

The foregoing detailed description has set forth various embodiments of the devices and/or processes via the use of flowcharts, diagrams, figures and/or examples.

Insofar as such flowcharts, diagrams, figures and/or examples contain one or more functions and/or operations, it will be understood by those within the art that each function and/or operation within such flowchart, diagram, figure and/or example can be implemented, individually and/or collectively, by a wide range of any combination thereof.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

The herein described subject matter sometimes illustrates different components contained within, or connected with, different other components. It is to be understood that such depicted figures are merely exemplary, and that in fact many other figures can be implemented which achieve the same functionality. In a conceptual sense, any arrangement of components to achieve the same functionality is effectively “associated” or “coupled” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to, physically mateable and/or physically interacting components and/or wirelessly interactable and/or wirelessly interacting components and/or logically interacting and/or logically interactable components.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to describe devices and/or processes in the fashion set forth herein, and thereafter use engineering practices to integrate such described devices and/or processes into image processing systems. That is, at least a portion of the devices and/or processes described herein can be integrated into an image processing system via a reasonable amount of experimentation. Those having skill in the art will recognize that a typical image processing system generally includes one or more of a system unit housing, a video display device, a memory such as volatile and non-volatile memory, processors such as microprocessors and digital signal processors, computational entities such as operating systems, drivers, and applications programs, one or more interaction devices, such as a touch pad or screen, control systems including feedback loops and control motors (e.g., feedback for sensing lens position and/or velocity; control motors for moving/distorting lenses to give desired focuses). A typical image processing system may be implemented utilizing any suitable commercially available components, such as those typically found in digital still systems and/or digital motion systems.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that a limitation is desired.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “operably coupled” or “coupled” or “in communication with” or “communicates with” or “operatively communicate” such other objects that the desired functionality is achieved. Hence, any two components herein combined to achieve a particular functionality can be seen as associated with each other such that the desired functionality is achieved, irrespective of architectures or intermedial components. Likewise, any two components so associated can also be viewed as being “connected”, or “attached”, to each other to achieve the desired functionality, and any two components capable of being so associated can also be viewed as being “operably couplable”, to each other to achieve the desired functionality.

While particular aspects of the present subject matter described herein have been shown and described, it will be apparent to those skilled in the art that, based upon the embodiments herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., ” a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., ” a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

1. A medical device comprising:
a tube portion shaped and sized for airway passage insertion and having at least one pathway therethrough;
at least one stylet having at least one shape-transforming material, configured for insertion into one of said at least one pathway; and
at least one sensor carried by one of said tube portion and/or by one of said at least one stylet.

2. The medical device of claim 1, wherein said tube portion includes a plurality of pathways therethrough.

3-6. (canceled)

7. The medical device of claim 1, wherein said tube portion includes at least one sensor that is located in a proximity to an end of said tube portion.

8. The medical device of claim 1, wherein said tube portion includes multiple sensors.

9. The medical device of claim 1, further comprising a source of illumination.

10-11. (canceled)

12. The medical device of claim 9, wherein said source of illumination includes at least one of an ultrasonic source, an acoustic source, a visible source, an ultraviolet source, a gamma ray source, an X-ray source and/or an infrared source.

13. The medical device of claim 1, further comprising at least one data-transmission device disposed in or outside one of said tube portion, said at least one data-transmission device being configured to operatively communicate with said at least one sensor.

14-16. (canceled)

17. The medical device of claim 9, wherein said illumination source is operably coupled to at least one image-acquisition device, and said image-acquisition device includes at least one of a camera, a charge coupled device, an X-ray receiver, an acoustic energy receiver, an electromagnetic energy receiver and/or an imaging device.

18. The medical device of claim 9, wherein said illumination source is operably coupled to at least one image-acquisition device, and said image-acquisition device is wirelessly coupled to at least one visual display.

19-32. (canceled)

33. The medical device of claim 1, wherein said tube portion is pliable.

34. The medical device of claim 1, wherein said tube portion is malleable.

35-36. (canceled)

37. The medical device of claim 1, wherein said tube portion is deformable.

38. The medical device of claim 1, wherein one of said at least one stylet is deformable.

39-48. (canceled)

49. The medical device of claim 1, wherein at least a portion of one of said at least one stylet is non-cylindrically shaped.

50-53. (canceled)

54. The medical device of claim 1, wherein said at least one sensor is disposed outside said tube portion.

55. The medical device of claim 1, wherein said at least one sensor is located proximate to a first end of one of said at least one stylet.

56. The medical device of claim 1, wherein said at least one sensor is located proximate to a first end of said tube portion.

57-70. (canceled)

71. The medical device of claim 1, wherein said tube portion includes one or more lumens and/or channels adapted for independent lung ventilation.

72. The medical device of claim 1, wherein one of said at least one stylet is integrally formed with said at least one shape-transforming material.

73. The medical device of claim 1, wherein one of said at least one stylet is made from said at least one shape-transforming material.

74. The medical device of claim 1, wherein said at least one shape-transforming material is made from a shape memory alloy.

75. The medical device of claim 74, wherein said shape memory alloy includes at least one of titanium, nickel, zinc, copper, aluminum, cadmium, platinum, iron, manganese, cobalt, gallium and/or tungsten.

76. The medical device of claim 74, wherein said shape memory alloy includes Nitinol™.

77. The medical device of claim 1, wherein said at least one shape-transforming material includes electro-active polymer.

78-79. (canceled)

80. The medical device of claim 1, wherein one of said at least one stylet is guided and/or actuated through one of said at least one pathway by an application of a voltage profile.

81-85. (canceled)

86. The medical device of claim 1, wherein one of said at least one stylet is guided and/or actuated through one of said at least one pathway by a solid-state phase change.

87. The medical device of claim 1, wherein one of said at least one stylet is guided and/or actuated through one of said at least one pathway by interaction with at least a portion of a wall of said one pathway.

88. The medical device of claim 87, wherein said guidance and/or actuation of one of said at least one stylet through one of said at least one pathway by interaction with at least a portion of a wall of said one pathway includes said guidance and/or actuation through said one pathway in response to separation between said at least a portion of said stylet and at least a portion of said wall.

89-91. (canceled)

92. The medical device of claim 1, wherein said tube portion shaped and sized for airway passage insertion includes said tube portion being configured for co-navigation through said airway passage.

93-255. (canceled)

256. A method of airway intubation comprising:
capturing an image adjacent to an airway tube proximate to an airway passage;
imaging said airway passage using at least one modality to capture images of anatomical structures adjacent to said airway passage; and
continually imaging over periods of time said airway passage and said anatomical structures adjacent to said airway passage.

257. The method of claim 256, further comprising: actuating, in response to said image, at least one shape-transformation of said airway tube.

258. The method of claim 257, wherein said actuating, in response to said image, at least one shape-transformation of said airway tube includes actuating a shape memory alloy disposed in said airway tube.

259. The method of claim 257, wherein said actuating, in response to said image, at least one shape-transformation of said airway tube includes a steady-state configuration of said airway tube in a fixed direction during an imaging procedure by using one or more force sensors and/or by using one or more devices for adjusting said configuration of said airway tube.

260. The method of claim 257, wherein said actuating, in response to said image, at least one shape-transformation of said airway tube includes guiding said airway tube through said airway passage by actuating at least one shape-transformation of said airway tube

261. The method of claim 257, wherein said actuating, in response to said image, at least one shape-transformation of said airway tube includes actuating an ex-vivo shape-transformation by an application of at least one of temperature, electricity, electromagnetic energy, magnetic force, microwave energy, acoustic energy or pressure on said airway tube.

262. The method of claim 257, wherein said actuating, in response to said image, at least one shape-transformation of said airway tube includes an in-vivo shape-transformation by an application of at least one of temperature, electricity, electromagnetic energy, magnetic force, microwave energy, acoustic energy or pressure on said airway tube.

263. The method of claim 257, wherein said actuating, in response to said image, at least one shape-transformation of said airway tube includes applying one or more temperature cycles or temperature profiles on said airway tube.

264. The method of claim 257, wherein said actuating, in response to said image, at least one shape-transformation of said airway tube includes steering said airway tube in a direction coincident with an airway.

265. The method of claim 256, wherein said continually imaging over periods of time includes capturing a plurality of images from an airway tube placed in an airway during prolonged airway intubation.

266-273. (canceled)

274. The method of claim 256, further comprising inserting a stylet.

275. The method of claim 274, wherein said inserting a stylet includes inserting said stylet having at least one shape-transforming material.

276. The method of claim 274, wherein said inserting a stylet includes inserting said stylet having one or more sensors into said airway tube.

277. The method of claim 276, wherein said at least one of said one or more sensors are adapted for transmitting information.

278-286. (canceled)
